(19) United States
(12) Patent Application Publication
(10) Pub. No.: US 2021/0205322 A1
(43) Pub. Date: Jul. 8, 2021

(54) RICTOR-TARGETED THERAPY IN THE MANAGEMENT OF BRAIN METASTASES

(71) Applicants: Albert Einstein College of Medicine, Bronx, NY (US); Montefiore Medical Center, Bronx, NY (US)

(72) Inventors: Haiying Cheng, Scarsdale, NY (US); Roman Perez-Soler, New York, NY (US); Balazs Halmos, Ridgefield, CT (US); Ni Fan, Shanghai (CN); Yiyu Zou, Bronx, NY (US)

(21) Appl. No.: 17/144,715

(22) Filed: Jan. 8, 2021

Related U.S. Application Data
(60) Provisional application No. 62/958,427, filed on Jan. 8, 2020.

(51) Int. Cl. A61K 31/5377; A61P 35/00; A61P 35/04; A61K 31/519; A61K 31/426; A61K 31/517; A61K 31/506; A61K 31/53; A61K 31/4545; A61K 31/47; A61K 31/4745; A61K 31/4985; A61K 31/496; A61K 31/437

(52) U.S. Cl. CPC A61K 31/5377; A61P 35/00; A61P 35/04; A61K 31/519; A61K 31/426; A61K 31/517; A61K 31/437; A61K 31/53; A61K 31/4545; A61K 31/47; A61K 31/4745; A61K 31/4985; A61K 31/496; A61K 31/506

(57) ABSTRACT

A method of treating metastases (e.g., brain metastases) in a patient with cancer (e.g., lung cancer) and at risk for metastases, exhibiting symptoms of metastases, or identified with metastases includes administering a therapeutically effective amount of a RICTOR inhibitor for the treatment of metastases. A method of reducing resistance to an EGFR, MET or AKT inhibitor in a cancer patient being administered the EGFR. MET or AKT inhibitor comprises co-administering a therapeutically effective amount of a RICTOR inhibitor and the EGFR, MET or AKT inhibitor. RICTOR inhibitors include compounds of formulas I-IV.

### CROSS-REFERENCE TO RELATED APPLICATIONS

This application claims priority to U.S. Provisional Application 62/958,427 filed on Jan. 8, 2020, which is incorporated herein by reference in its entirety.

### STATEMENT REGARDING FEDERALLY SPONSORED RESEARCH & DEVELOPMENT

This invention was made with government support 5K12 CA132783-04 awarded by the National Institutes of Health (NIH)/National Cancer Institute (NCI). The government has certain rights in the invention.

### FIELD OF THE DISCLOSURE

The present disclosure is related to compositions and methods for the treatment of metastases, particularly brain metastases associated with lung cancer, and reducing resistance to molecularly-targeted therapy such as EGFR therapy.

### BACKGROUND

Both cancer metastasis and treatment resistance are major contributors of cancer death. Approximately 20-50% of advanced lung cancer patients develop brain metastases and have a very dismal prognosis. Current treatment modalities offer only a modest clinical benefit with significant risks and long-term adverse effects. At the same time, patients with Epidermal Growth Factor Receptor (EGFR)-mutant lung cancer usually respond well to EGFR tyrosine kinase inhibitors (TKI) initially, but subsequently develop resistance and ultimately fail the treatment, presenting a serious clinical problem.

What is needed are new therapies for the treatment and prevention of brain metastases that also have the potential to reduce resistance to EGFR inhibitors.

### BRIEF SUMMARY

In one aspect, a method of treating or preventing metastases (e.g., brain metastases) in a patient with cancer (e.g., lung cancer) and at risk for metastases, exhibiting symptoms of metastases, or identified with metastases, comprises administering a therapeutically effective amount of a RICTOR inhibitor for the treatment of metastases.

In another aspect, a method of reducing resistance to an EGFR, ALK or MET inhibitor in a cancer patient being administered the EGFR, ALK or MET inhibitor comprises co-administering a therapeutically effective amount of a RICTOR inhibitor and the EGFR, ALK or MET inhibitor.

Inhibitors targeting the RICTOR signaling pathways (called RICTOR inhibitors) as used herein are selected from:

a) a compound having the structure of formula I or a pharmaceutically acceptable salt thereof:

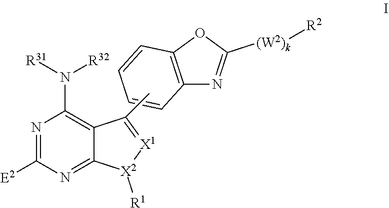

wherein:

X¹ is N or C-E¹ and X² is N; or X¹ is NH or CH-E¹and X² is C;

R¹ is hydrogen, -L-C₁-C₁₂alkyl, -L-C₃-C₈cycloalkyl, -L-C₁-C₁₂alkyl-C₃-C₈cycloalkyl, -L-aryl, -L-heteroaryl, -L-heterocyclyl, -L-C₁-C₁₂alkylheteroaryl, -L-C₁-C₁₂alkylheterocyclyl, wherein each R¹ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂, NR³¹R³², —CO₂R³¹, CO₂aryl, —C(═O)NR³¹R³², —NO₂, or —CN;

L is absent, —(C═O)—, —C(═O)O—, —C(═O)N(R³¹)—, —S—, —S(═O)—, —S(═O)₂—, —S(═O)₂N(R³¹)—, or —N(R³¹);

k is 0 or 1;

E¹ and E² are independently —(W¹)ⱼ—R³⁴;

j in E¹ or j in E², is independently 0 or 1;

W¹ is —O—, —NR⁷—, —S(═O)₀₋₂—, —C(═O)—, —C(═O)N(R⁷)—, —N(R⁷)C(═O)—, —N(R⁷)S(═O)—, 13 N(R⁷)S(═O)₂—, —C(══O)O—, —CH(R⁷)N(C(═O)OR⁸)—, —CH(R⁷)N(C(═O)R⁸)—, —CH(R⁷)NS(═O)₂O—, —CH(R⁷)N(R⁸)—, —CH(R⁷)C(═O)N(R⁸)—, —CH(R⁷)N(R⁸)C(═O)—, —CH(R⁷)N(R⁸)S(═O)—, or —CH(R⁷)N(R⁸)S(═O)₂—;

W² is —O—, —NR⁷, —S(═O)₀₋₂—, —C(═O)—, —C(═O)N(R⁷)—, —N(R⁷)C(═O)—, —N(R⁷)C(═O)N(R⁸)—, —N(R⁷)S(═O)—, —N(R⁷)S(═O)₂—, —C(═O)O—, —CH(R⁷)N(C(═O)OR⁸)—, —CH(R⁷)N(C(═O)R⁸)—, —CH(R⁷)N(SO₂R⁸)—, —CH(R⁷)N(R⁸)—, —CH(R⁷)C(═O)N(R⁸)—, —CH(R⁷)N(R⁸)C(═O)—, —CH(R⁷)N(R⁸)S(═O)—, or —CH(R⁷)N(R⁸)S(═O)₂—;

R² is hydrogen, halogen, —OH, —R³¹, —CF₃, —OCF₃, —OR³¹, —NR³¹R³²,—C(═O)R³¹, —CO₂R³¹, —C(═O)NR³¹R³²,—NO₂, —CN, aryl, heteroaryl, C₁-C₁₂ alkyl, C₁-C₁₂haloalkyl, or C₃-C₈cycloalkyl, wherein each of the aryl or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

R³⁴ is hydrogen, halogen, —OH, —R³¹, —CF₃, —OCF₃, —OR³¹, —NR³¹R³², —C(═O)R³¹, —CO₂R³¹, —C(═O)NR³¹R³², —NO₂, —CN, —NR³¹C(═O)R³², —NR³¹C(═O)OR³², aryl, heteroaryl, heterocyclyl, C₁-C₁₂ alkyl, C₁-C₁₂ haloalkyl, or C₃-C₈cycloalkyl, wherein each of the aryl, heteroaryl, or heterocyclyl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

each R³¹ and R³² independently is hydrogen or C₁-C₁₂alkyl, wherein the C₁-C₁₂alkyl is optionally substituted with aryl, heterocyclyl, or heteroaryl group, wherein each of the aryl, heterocyclyl, or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN; and

each R⁷ and R⁸ independently is hydrogen, C₁-C₁₂alkyl, aryl, heteroaryl, heterocyclyl or C₃-C₁₀cycloalkyl, wherein each R⁷ and R⁸ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

b) a compound having the structure of formula II or a pharmaceutically acceptable salt thereof:

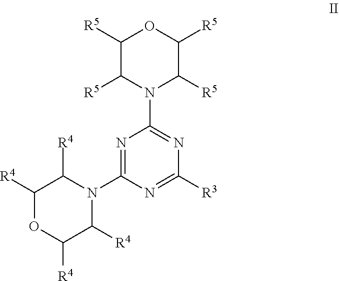

wherein:

R³ is an aryl or heteroaryl group, specifically pyridyl, pyridazine, pyrimidine, or pyrazine, wherein R³ is optionally substituted with 1, 2, or 3 substituents, each substituent independently is halogen, C₁-C₁₂ alkyl, C₁-C₁₂ haloalkyl, C₁-C₁₂ alkoxy, —NR₁₀R¹¹, —OR¹⁰, —C(═O)R¹⁰, —NR¹⁰C(═O)R¹¹, —N(C(═O)R¹¹)₂, —NR¹⁰C(═O)NR¹⁰R¹¹, —C(═O)OR¹⁰, or —C(═O)NR¹⁰R¹¹;

each R⁴ and R⁵ are independently hydrogen, halogen, or C₁-C₁₂ alkyl, specifically hydrogen or C₁-C₂ alkyl;

each instance of R¹⁰ and R¹¹ independently is hydrogen, C₁-C₁₂ alkyl, or C₃-C₁₂ carbocyclyl, or

R¹⁰ and R¹¹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;

c) a compound having the structure of formula III or a pharmaceutically acceptable salt thereof:

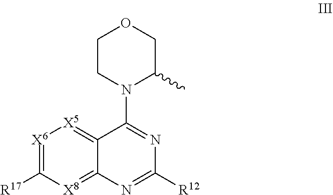

wherein:

one or two of X⁵, X⁶ and X⁸ is N, and the others are CH;

R¹⁷ is hydrogen, halogen, C₁-C₁₂ alkyl, C₁-C₁₂ haloalkyl, OR¹⁰, SR¹⁰, NR¹⁰R¹¹, aryl, or heteroaryl, wherein the aryl and heteroaryl are optionally substituted with 1, 2, or 3 substituents, each substituent independently is halogen, C₁-C₁₂ alkyl, C₁-C₁₂ haloalkyl, C₁-C₁₂ alkoxy, —NR¹⁰R₁₁, —OR¹⁰, —C(═O)R¹⁰, —NR¹⁰C(═O)R¹¹, —N(C(═O)R¹¹)₂, —NR¹⁰C(═O)NR¹⁰R¹¹, —C(═O)OR¹⁰, or —C(═O)NR¹⁰R¹¹;

each instance of R¹⁰ and R¹¹ independently is hydrogen, C₁-C₁₂ alkyl, or C₃-C₁₂ carbocyclyl, or

R¹⁰ and R¹¹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;

R¹² is hydrogen, halogen, C₁-C₁₂ alkyl, C₁-C₁₂ haloalkyl, OR¹⁰, SR¹⁰, NR¹⁸R¹⁹, aryl, or heteroaryl;

R¹⁸ and R¹⁹ are independently hydrogen, C₁-C₁₂ alkyl, or C₃-C₁₂ carbocyclyl, or

R¹⁸ and R¹⁹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, specifically R¹⁸ and R¹⁹ together with the nitrogen to which they are attached form an optionally substituted morpholino; or

d) a compound having the structure of formula IV or a pharmaceutically acceptable salt thereof:

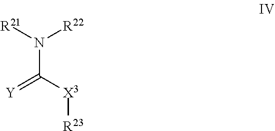

wherein:

R²¹ is aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

R²² is C₁-C₁₂alkyl, aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

R²³ is C₁-C₁₂alkyl, aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

each R³¹ and R³² independently is hydrogen or C₁-C₁₂alkyl, wherein the C₁-C₁₂alkyl is optionally substituted with aryl, heterocyclyl, or heteroaryl group, wherein each of the aryl, heterocyclyl, or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

X³ is C(R⁴¹R⁵¹), N(R⁴¹), or O, wherein each R⁴¹ and R⁵¹ independently is hydrogen or C₁-C₁₂ alkyl, specifically X³ is N(R⁴¹), more specifically X³ is N(H); and

Y is S or O, specifically O.

### BRIEF DESCRIPTION OF THE DRAWINGS

FIGS. 1A-J show RICTOR inhibition can restore the EGFR TKI sensitivity in TKI resistant EGFR-mutant lung cancer cells. 1A) Western blot analysis and 1B) MTS cell viability test in the HCC827 parental cells and EGFR TKI erlotinib-resistant cells (ER3) with erlotinib treatment. 1C) Western blot analysis and 1D) MTS cell viability assay in the doxycycline-inducible RICTOR knockdown ER3 cells. 1E) Clonogenic survival assay in the ER3 cells with genetically RICTOR knockdown and 1F) pharmacological inhibitor, saparnisertib (10 nM). 1G) ER3 mouse xenograft tumor growth model with treatment of doxycycline and erlotinib. 1H) Western blot analysis using ER3 mouse model tumor lysate. 3I) MTS cell viability assay and 1J) clonogenic survival assay in the ER3 cells with afatinib and osimertinib treatments.

FIGS. 2A-C show RICTOR upregulation is a common resistant mechanism in EGFR-mutant lung cancer cells. 2A) Western blot analysis of inducible upregulated RICTOR HCC827 (HCC827 OE). 2B) RICTOR-dependent tumor cell growth identified by cell proliferation assay. 2C) Clonogenic survival assay of HCC827 OE treated with erlotinib, afatinib and osimertinib.

FIGS. 3A-D show RICTOR inhibition promotes the sensitivity of lung cancer cells. 3A and 5B) Drug response in the HCC827 R4 and PC-9 cells were evaluated by clonogenic survival assay. 3C) The effect of combined treatment of TKIs and saparnisertib (5 nM) were detected in PC9 parental cells. 3D) HCC827 R4 tumor xenograft model with treatment of doxycycline and erlotinib.

FIGS. 4A-E show RICTOR drives TKI resistance by negatively regulating Axl degradation. 4A) Western blot analysis in the ER3 R4, PC9 R4 and HCC827 R4 cells with erlotinib treatment (1 μM, 10 nM and 1 nM respectively). 4B) Secreted caspase-3 level was evaluated by colorimetric assay. The erlotinib and sapanisertib concentration for ER3 R4 was 10 μM and 10 nM, and for PC9 R4 is 8 nM and 2 nM. 4C) Western blot analysis of HCC827 OE with erlotinib treatment (1 nM). 4D) Western blot analysis of ER3 R4 and HCC827 OE with osimertinib treatment (0.5 μM and 50 nM). 4E) The half-life of Axl in PC9 R4 cells was evaluated by using CHX.

FIGS. 5A-D show the upregulation of EMT may be involved in the RICTOR-mediated EGFR TKI resistance. 5A) Analysis of epithelial marker (E-cadherin) and mesenchymal marker (N-cadherin) in ER3 R4 and HCC827 OE cells. In vitro transwell cell migration and invasion assay was tested in 5B) HCC827 R4, 5C) PC9 R4 and 5D) HCC827 OE cells.

FIGS. 6A-C show the frequency of genetic alterations in primary tumor vs. brain metastases vs. other metastatic sites in lung adenocarcinoma (n=11845 cases). Among all tested genes in this pathway, RICTOR amplification is associated with the highest enrichment in brain metastases. 6A) PI3K/AKT/mTOR gene alterations in brain vs. primary. 6B) RICTOR amplification in brain vs. primary. 6C) PTEN, AKT1, PIK3CA or mTOR genetic alterations in brain vs. primary.

FIGS. 7A-C show inducible RICTOR knockdown in H23 lung cancer cells (parental line with RICTOR amplification) was associated with reduced cell migration and invasion, whereas upregulation of RICTOR in HCC827 lung cancer cells (parental line with normal RICTOR copies) was associated with an increase of both processes. 7A, B) Inducible RICTOR knockdown in H23 lung cancer cells reduces cell migration and invasion, whereas upregulation of RICTOR in HCC827 lung cancer cells promotes these processes. 7C) mTOR inhibitors significantly reduced migration and invasion in vitro in RICTOR-amplified H23 and H1703 lung cancer cells.

FIGS. 8A and B show in vivo metastasis assays using the stereotactic brain injection models. 8A) Inducible RICTOR knockdown (with addition of doxycycline through oral lavage daily) reduced the tumor growth of H23-R4-Luc in the brain. *=P<0.05. 8B) After 4 weeks of daily oral lavage, TAK228 significantly reduced tumor growth in the brain, including 43% (3 out 7) near complete responses.

FIG. 9 shows RICTOR ablation in H23 cells promoted the expression of E-cadherin (epithelial marker), suppressed the expression of N-cadherin and vimentin (mesenchymal markers), as well as decreased the level of pAKT and CXCR4 (a marker of brain metastasis). The opposite effects on these markers are noted with RICTOR upregulation in HCC827 cells.

FIGS. 10A-E shows both AKT inhibitors significantly decreased lung cancer cell invasion and migration, associated with increased apoptosis (as shown by elevated cleaved PARP).

FIG. 11A-G shows that RICTOR may regulate the brain metastasis process through AKT and CXCL12 chemokine-CXCR4 axis. 11A) shows inducible RICTOR knockdown resulted in less total CXCR4, and inhibited the activation of CXCR4 pathway with reduced p-CXCR4 and p-FAK. 11B and C show that activation of the CXCR4 pathway with treatment of SDF-1α led to increased p-FAK, (p-AKT), and p-S6, along with increased migration and invasion while, AMD3100, a pharmacological CXCR4 inhibitor, significantly reduced SDF-1α-induced invasion and migration, through blocking CXCR4/FAK/S6 signaling. 11D-G show that the addition of CXCR4 siRNAs to inducible RICTOR knockdown led to further inhibition of CXCR4 signaling, more pronounced apoptosis, and significantly further reduced invasion and migration.

The above-described and other features will be appreciated and understood by those skilled in the art from the following detailed description, drawings, and appended claims.

### DETAILED DESCRIPTION

The inventors have previously reported that RICTOR amplification is a novel driver mutation in a subset of lung cancer patients. This result has been confirmed in lung cancer and in other types of tumors. Unexpectedly, the inventors found that RICTOR is particularly enriched in brain metastases, particularly brain metastases in advanced lung cell cancers. RICTOR also plays a role in mediating resistance to EGFR inhibitors, thus inhibitors targeting the RICTOR signaling pathways can also be used to prevent resistance to EGFR therapy secondary to activated RICTOR signaling.

Epidermal Growth Factor Receptor (EGFR) activated mutations occur in about 20% of non-small cell lung cancer patients (NSCLC). Currently, the EGFR tyrosine kinase inhibitors (TKIs) are the standard of care for the treatment of NSCLC patients harboring these mutations. Comparing to traditional chemotherapy, EGFR TKIs lead to better responses and longer survival in patients with activating EGFR mutations, including exon 19 deletions and L858R mutations. However, it is common for the patients to acquire treatment resistance. Several molecular mechanisms and bypass pathways of acquired resistance of first- and second-generation of TKIs have been identified, including the T790M secondary mutation in exon 20 (approximately 49%), MET amplification (approximately 5%), hepatocyte growth factor (HGF) overexpression and PIK3CA mutation (approximately 5%). The third generation of EGFR TKI osimertinib, targeting both T790M mediated acquired resistance and sensitizing EGFR mutations, has shown success with improved clinical outcomes in both front-line and second-line settings. As first-line treatment in EGFR mutated advanced NSCLC, osimertinib showed better efficacy superior to that of erlotinib or gefitinib with longer progression-free survival and overall survival. However, similar to previous TKIs, patients receiving osimertinib also inevitably develop treatment resistance, such as MET amplification and D1228V mutation, and EGFR C797S mutation, following by HER-amplification, PIK3CA, and RAS mutations. In addition, recent studies have proposed that activated Axl signaling is a new mechanism of osimertinib resistance in EGFR mutant lung cancer. Combining an Axl inhibitor with osimertinib could be a new strategy to overcome osimertinib resistance. Despite all these interesting findings, drug resistance remains a major challenge for all the EGFR TKIs. A further understanding of intrinsic and acquired drug resistance of TKIs in EGFR mutated NSCLC is needed for the further clinical application of these inhibitors.

RICTOR is a key component of the mTORC2 complex in the PI3K/AKT/mTOR signaling pathway. It plays a critical role in mediating cellular homeostasis, actin reorganization and cell survival. The most well-established function of RICTOR/mTORC2 signaling is to fully activate AKT by phosphorylating AKT at Serine 473. RICTOR also has other substrates, mainly AGC kinases. RICTOR is also involved in mTORC2-independent biological signaling pathways, highlighting its potential important functions in regulating cell activities, especially tumor growth, progression and metastasis. In several in vitro and in vivo cancer models, RICTOR has been suggested to drive tumor proliferation and drug resistance. RICTOR amplification has been reported in around 10% of lung cancer patients, and often concurrent with other oncogenic mutations, such as EGFR alteration and KRAS mutations. The functional roles of RICTOR in these driver-mutated lung cancers remain elusive, warranting further studies.

Here the inventors show that RICTOR activation is associated with EGFR TKI resistance in EGFR mutant NSCLC and it appears to be a common resistance mechanism with all three generations of EGFR TKIs, including osimertinib. The combined treatment of RICTOR inhibition and EGFR TKIs effectively restored the sensitivity in both resistant cells and tumor xenografts. In addition, the inventors identified a novel functional role of RICTOR to control Axl stability. By downregulating Axl degradation and promoting EMT, activation of RICTOR signaling leads to EGFR TKI resistance. RICTOR amplification could be a novel resistance mechanism to EGFR TKIs, thus co-targeting RICTOR and EGFR may be a novel therapeutic strategy to overcome the treatment resistance in EGFR-mutated lung cancer.

In an aspect, a method of treating or preventing metastases (e.g., brain metastases) in a patient with cancer (e.g., lung cancer) and at risk for metastases, exhibiting symptoms of metastases, or identified with metastases comprises administering a therapeutically effective amount of a RICTOR inhibitor for the treatment of metastases. Inhibitors targeting the RICTOR signaling pathways (RICTOR inhibitors) include compounds of formulas I-IV as described herein.

Metastases such as brain metastases occur when cancer cells from a primary site such as the lung spread to another site such as the brain. Brain metastases are different from primary brain tumors which start in the brain. Cancers with a particular risk of brain metastases include lung cancer, breast cancer, colon cancer, kidney cancer and melanoma. A particular type of lung cancer associated with brain metastases is non-small cell lung cancer, specifically advanced non-small cell lung cancer.

Exemplary metastases include brain, liver, kidney, bone, adrenal gland, lymph node, and pleural metastases. In an aspect, the metastases are brain metastases.

In an aspect, the method comprises, prior to administering, identifying brain metastases in the patient. Magnetic resonance imaging (MRI), computerized tomography (CT), and/or positron emission tomography (PET) can be used to identify brain metastases.

In an aspect, the patient is exhibiting symptoms of brain metastases. Symptoms of brain metastasis associated with cancer include changes in cognitive ability such as decreases in memory, attention and reasoning; headaches; weakness, nausea; vomiting; dizziness; balance issues; difficult speaking; numbness; tingling sensations; behavioral and personality changes; difficulty swallowing; seizures; and combinations thereof.

In another aspect, method of reducing resistance to an EGFR inhibitor in a cancer patient being administered the EGFR inhibitor comprises co-administering a therapeutically effective amount of a RICTOR inhibitor and an EGFR inhibitor. Inhibitors targeting the RICTOR signaling pathways of formulas I-IV are described in detail below.

In an aspect, the patient exhibits an EGFR mutation such as a T790M secondary mutation in exon 20, MET amplification, hepatocyte growth factor (HGF) overexpression and/or a PIK3CA mutation.

In an aspect, the EGFR inhibitor is a first generation EGFR inhibitor such as gefitinib, icotinib and erlotinib.

In an aspect, the EGFR inhibitor is a second generation EGFR inhibitor such as afatinib and dacomitinib.

In an aspect, the EGFR inhibitor is a third generation EGFR inhibitor such as osimertinib, olmutinib, PF-06747775, YH5448, avitinib and rociletinib.

In an aspect, the RICTOR inhibitor and EGFR inhibitor are not sapanisertib and osimertinib.

In another aspect, method of reducing resistance to a MET inhibitor in a cancer patient being administered the MET inhibitor, comprising co-administering a therapeutically effective amount of a RICTOR inhibitor and a MET inhibitor. Inhibitors targeting the RICTOR signaling pathways of formulas I-IV are described in detail below.

Exemplary MET inhibitors include capmatinib, tepotinib, crizotinib, cabozantinib, foretinib, tibantinib, and savolitinib.

In another aspect, method of reducing resistance to an ALK inhibitor in a cancer patient being administered the ALK inhibitor comprises co-administering a therapeutically effective amount of a RICTOR inhibitor and an EGFR inhibitor. Inhibitors targeting the RICTOR signaling pathways of formulas I-IV are described in detail below.

Exemplary ALK inhibitors include crizotinib, alectinib, brigatinib, ceritinib and lorlatinib.

In one aspect a RICTOR inhibitor is a compound having the structure of formula I or a pharmaceutically acceptable salt thereof:

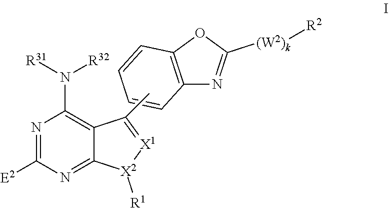

wherein:

X¹ is N or C-E¹ and X² is N; or X¹ is NH or CH-E¹ and X² is C;

R¹ is hydrogen, -L-C₁-C₁₂alkyl, -L-C₃-C₈cycloalkyl, -L-C₁-C₁₂alkyl-C₃-C₈cycloalkyl, -L-aryl, -L-heteroaryl, -L-heterocyclyl, -L-C₁-C₁₂alkylaryl, -L-C₁-C₁₂alkylheteroaryl, -L-C₁-C₁₂alkylheterocyclyl, wherein each R¹ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

L is absent, —(C═O)—, —C(═O)O—, —C(═O)N(R³¹)—, —S—, —S(═O)—, —S(═)₂—, —S(═O)₂N(R³¹)—, or —N(R³¹);

k is 0 or 1;

E¹ and E² are independently —(W¹)ⱼ—R³⁴;

j in E¹ or j in E², is independently 0 or 1;

W¹ is —O—, —NR⁷—, —S(═O)₀₋₂—, —C(═O)—, —C(═O)N(R⁷)—, —N(R⁷)C(═O—, —N(R⁷)S(═O)—, —N(R⁷)S(═O)₂—, —C(══O)O—, —CH(R⁷)N(C(═O)OR⁸)—, —CH(R⁷)N(C(═O)R⁸)—, —CH(R⁷)NS(═O)₂O—, —CH(R⁷)N(R⁸)—, —CH(R⁷)C(═O)N(R⁸)—, —CH(R⁷)N(R⁸)C(═O)—, —CH(R⁷)N(R⁸)S(═O)—, or —CH(R⁷)N(R⁸)S(═O)₂—;

W² is —O—, —NR⁷, —S(═O)₀₋₂—, —C(═O)—, —C(═O)N(R⁷)—, —N(R⁷)C(═O)—, —N(R⁷)C(═O)N(R⁸)—, —N(R⁷)S(═O)—, —N(R⁷)S(═O)₂—, —C(═O)O—, —CH(R⁷)N(C(═O)OR⁸)—, —CH(R⁷)N(C(═O)R⁸)—, —CH(R⁷)N(SO₂R⁸)—, —CH(R⁷)N(R⁸)—, —CH(R⁷)C(═O)N(R⁸)—, —CH(R⁷)N(R⁸)C(═O)—, —CH(R⁷)N(R⁸)S(═O)—, or —CH(R⁷)N(R⁸)S(═O)₂—;

R² is hydrogen, halogen, —OH, —R³¹, —CF₃, —OCF₃, —OR³¹, —NR³¹R³²,—C(═O)R³¹, —CO₂R³¹, —C(═O)NR³¹R³²,—NO₂, —CN, aryl, heteroaryl, C₁-C₁₂alkyl, C₁-C₁₂haloalkyl, or C₃-C₈cycloalkyl, wherein each of the aryl or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

R³⁴ is hydrogen, halogen, —OH, —R³¹, —CF₃, —OCF₃, —OR³¹, —NR³¹R³², —C(═O)R³¹, —CO₂R³¹, —C(═O)NR³¹R³², —NO₂, —CN, —NR³¹C(═O)R³², —NR³¹C(═O)OR³², aryl, heteroaryl, heterocyclyl, C₁-C₁₂alkyl, C₁-C₁₂haloalkyl, or C₃-C₈cycloalkyl, wherein each of the aryl, heteroaryl, or heterocyclyl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

each R³¹ and R³² independently is hydrogen or C₁-C₁₂alkyl, wherein the C₁-C₁₂alkyl is optionally substituted with aryl, heterocyclyl, or heteroaryl group, wherein each of the aryl, heterocyclyl, or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R₃₂, —NO₂, or —CN; and each R⁷ and R⁸ independently is hydrogen, C₁-C₁₂alkyl, aryl, heteroaryl, heterocyclyl or C₃-C₁₀cycloalkyl, wherein each R⁷ and R⁸ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN.

In a further aspect, the compound is according to the structure of formula I, wherein

X¹ is N and X² is N;

R¹ is hydrogen, -L-C₁-C₁₂alkyl, -L-C₃-C₈cycloalkyl, or -L-aryl, wherein each R¹ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO2R³¹, —CO₂aryl, —C(═O)NR³¹R³², —NO₂, or —CN;

L is absent;

k is 0;

E² is —(W¹)ⱼ—R³⁴;

j is 0;

R² is hydrogen, halogen, —OH, —R³¹, —CF₃, —OCF₃, —OR³¹, —NR³¹R³²,—C(═O)R³¹, —CO₂R³¹, —C(═O)NR³¹R³²,—NO₂, —CN, aryl, heteroaryl, C₁-C₁₂alkyl, C₁-C₁₂haloalkyl, or C₃-C₈cycloalkyl;

R³⁴ is hydrogen, halogen, —OH, —R³¹, —CF₃, —OCF₃, —OR³¹, —NR³¹R³², —C(═O)R³¹, —CO₂R³¹, —C(═O)NR³¹R³², —NO₂, —CN, aryl, heteroaryl, heterocyclyl, C₁-C₁₂alkyl, C₁-C₁₂haloalkyl, or C₃-C₈cycloalkyl; and each R³¹ and R³² independently is hydrogen or C₁-C₁₂alkyl.

Within the structure of formula I, the compound can be Sapanisertib (TAK-228 or MLN028; 5-(4-amino-1-propan-2-yl-3-pyrazolo[3,4-d]pyrimidinyl)-1,3-benzoxazol-2-amine) or the benzoxazole derivatives disclosed in WO2010/051043A1, the compounds and their synthesis of which are hereby incorporated herein in their entirety.

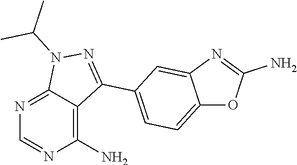 [SMILES: CC(C)n1nc(-c2ccc3oc(N)nc3c2)c2c(N)ncnc21]

5-(4-Amino-1-propan-2-yl-3-pyrazolo[3,4-d]pyrimidinyl)-1,3-benzoxazol-2-amine.

In one aspect a RICTOR inhibitor is a compound having the structure of formula II or a pharmaceutically acceptable salt thereof:

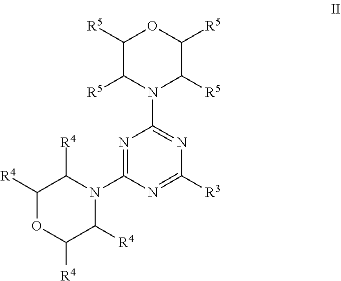

wherein:

R³ is an aryl or heteroaryl group, specifically pyridyl, pyridazine, pyrimidine, or pyrazine, wherein R³ is optionally substituted with 1, 2, or 3 substituents, each substituent independently is halogen, C₁-C₁₂ alkyl, C₁-C₁₂haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)₂, —NR¹⁰C(═O)NR¹⁰R¹¹, —C(═O)OR¹⁰, or —C(═O)NR¹⁰R¹¹;

each R⁴ and R⁵ are independently hydrogen, halogen, or C₁-C₁₂ alkyl, specifically hydrogen or C₁-C₂ alkyl;

each instance of R¹⁰ and R¹¹ independently is hydrogen, C₁-C₁₂ alkyl, or C₃-C₁₂ carbocyclyl, or

R¹⁰ and R¹¹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl.

In a further aspect, the compound is according to the structure of formula II, wherein

R³ is pyridyl, pyridazine, pyrimidine, or pyrazine, where R³ is optionally substituted with 1 or 2 substituents, each substituent independently is halogen, C₁-C₁₂ alkyl, C₁-C₁₂ haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)₂, —NR¹⁰C(═O)NR¹⁰R¹¹, —C(⊚O)OR¹⁰, or —C(═O)NR¹⁰R¹¹;

each R⁴ and R⁵ are independently hydrogen or C₁-C₂ alkyl; and

each instance of R¹⁰ and R¹¹ independently is hydrogen or C₁-C₁₂ alkyl.

Within the structure of formula II, the compound can be bimiralisib (PQR309; 5-(4,6-dimorpholin-4-yl-1,3,5-triazin-2-yl)-4-(trifluoromethyl)pyridin-2-amine) or the triazine derivatives disclosed in U.S. Pat. No. 8,921,361, the compounds and their synthesis of which are hereby incorporated herein in their entirety.

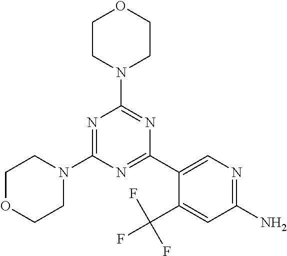 [SMILES: Nc1cc(C(F)(F)F)c(-c2nc(N3CCOCC3)nc(N3CCOCC3)n2)cn1]

5-(4,6-Dimorpholin-4-yl-1,3,5-triazin-2-yl)-4-(trifluoromethyl)pyridin-2-amine.

In one aspect a RICTOR inhibitor is a compound having the structure of formula III or a pharmaceutically acceptable salt thereof:

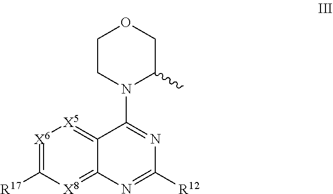

wherein:

one or two of X⁵, X⁶ and X⁸ is N, and the others are CH;

R¹⁷ is hydrogen, halogen, C₁-C₁₂ alkyl, C₁-C₁₂ haloalkyl, OR¹⁰, SR¹⁰, NR¹⁰R¹¹, aryl, or heteroaryl, wherein the aryl and heteroaryl are optionally substituted with 1, 2, or 3 substituents, each substituent independently is halogen, C₁-C₁₂ alkyl, C₁-C₁₂haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)₂, —NR¹⁰C(═O)NR¹⁰R¹¹, —C(═O)OR¹⁰, or —C(═O)NR¹⁰R¹¹,

each instance of R¹⁰ and R¹¹ independently is hydrogen, C₁-C₁₂ alkyl, or C₃-C₁₂ carbocyclyl, or

R¹⁰ and R¹¹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;

R¹² is hydrogen, halogen, C₁-C₁₂ alkyl, C₁-C₂haloalkyl, OR¹⁰, SR¹⁰, NR¹⁸R¹⁹, aryl, or heteroaryl;

R¹⁸ and R¹⁹ are independently hydrogen, C₁-C₁₂ alkyl, or C₃-C₁₂ carbocyclyl, or R¹⁸ and R¹⁹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, specifically R¹⁸ and R¹⁹ together with the nitrogen to which they are attached form an optionally substituted morpholino.

In a further aspect, the compound is according to the structure of formula III, wherein

X⁸is N and X⁵ and X⁶ are CH;

R¹² is NR¹⁸R¹⁹ wherein the R¹⁸ and R¹⁹ of NR¹⁸R¹⁹ together with the nitrogen to which they are attached form a C₃-C₂₀ heterocyclic ring containing one or more additional ring atoms N, O or S; and

R¹⁷ is aryl or heteroaryl, wherein the aryl and heteroaryl are optionally substituted with 1 or 2 substituents, each substituent independently is halogen, C₁-C₁₂ alkyl, C₁-C₁₂ haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)₂, —NR¹⁰C(═O)NR¹⁰R¹¹, —C(═O)OR¹⁰, or —C(═O)NR¹⁰R¹¹.

Within the structure of formula III, the compound can be Vistusertib (AZD2014); 3-[2,4-bis[(3S)-3-methylmorpholin-4-yl]pyrido[2,3-d]pyrimidin-7-yl]-N-methylbenzamide) or the derivatives disclosed in U.S. Pat. No. 7,902,189, the compounds and their synthesis of which are hereby incorporated herein in their entirety.

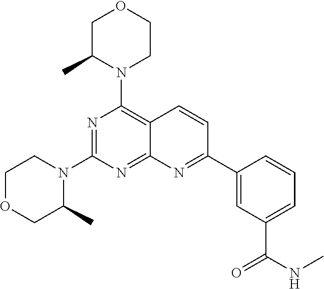 [SMILES: CNC(=O)c1cccc(-c2ccc3c(N4CCOC[C@@H]4C)nc(N4CCOC[C@@H]4C)nc3n2)c1]

3-[2,4-Bis [(3 S)-3-methylmorpholin-4-yl]pyrido[2,3-d]pyrimidin-7-yl]-N-methylbenzamide.

In one aspect a RICTOR inhibitor is compound having the structure of formula IV or a pharmaceutically acceptable salt thereof:

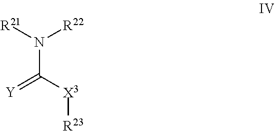

wherein:

R²¹ is aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂, —NR³¹R³², —CO₂R³¹, —CO₂aryl, —C(═O)NR³¹R³², —NO₂, or —CN;

R²² is C₁-C₁₂alkyl, aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

R²³ is C₁-C₁₂alkyl, aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

each R³¹ and R³² independently is hydrogen or C₁-C₁₂alkyl, wherein the C₁-C₁₂alkyl is optionally substituted with aryl, heterocyclyl, or heteroaryl group, wherein each of the aryl, heterocyclyl, or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;

X³ is C(R⁴¹R⁵¹), N(R⁴¹), or O, wherein each R⁴¹ and R⁵¹independently is hydrogen or C₁-C₁₂ alkyl, specifically X³ is N(R⁴¹), more specifically X³ is N(H); and

Y is S or O, specifically O.

Additional aspects of formula IV include: the compound of formula IV wherein R²¹ is 5-membered heteroaryl or heterocyclyl; the compound of formula IV wherein heterocyclyl includes optionally substituted 4,5-dihydrothiazol-2-yl, e.g., 5-methyl-4,5-dihydrothiazol-2-yl; he compound of formula IV wherein R²¹ is thiazolyl, isothiazolyl, oxazolyl, 4,5-dihydrooxazolyl, 4,5-dihydrothiazolyl, benzothiazolyl, benzoxazolyl, pyridyl, or phenyl; the compound of formula IV wherein R²² is optionally substituted phenyl, heteroaryl or heterocyclyl, specifically an alkyl substituted phenyl or halo substituted phenyl, e.g., 3,4-dimethylphenyl, 4-bromophenyl, or 4-fluorophenyl; the compound of formula IV wherein R²³ is optionally substituted phenyl, heteroaryl or heterocyclyl, and the compound of formula IV wherein R²³ is halo substituted phenyl, e.g., 3,4-dichlorophenyl.

Within the structure of formula IV, the compound can be CID613034 (3-(3,4-Dichlorophenyl)-1-(5-methyl-2-thiazolin-2-yl)-1-(3,4-xylyl)urea) or the derivatives disclosed in WO2018/187414A1, the compounds and their synthesis of which are hereby incorporated herein in their entirety.

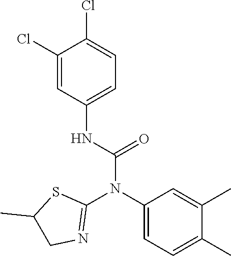

3-(3,4-Dichlorophenyl)-1-(5-methyl-2-thiazolin-2-yl)-1-(3,4-xyly)urea.

In certain aspects, the compounds of formulae I-IV may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with double bonds may occur in Z- and E-forms, with all isomeric forms of the compounds being included in the present disclosure. In these situations, the single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Compounds are described using standard nomenclature. For example, any position not substituted by any indicated group is understood to have its valency filled by a bond as indicated, or a hydrogen atom.

The term “substituted”, as used herein, means that any one or more hydrogens on the designated atom or group is replaced with a selection from the indicated group, provided that the designated atom's normal valence is not exceeded. When a substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“—”) that is not between two letters or symbols is used to indicate a point of attachment for a substituent. For example, —COOH is attached through the carbon atom.

As used herein, “alkyl” is intended to include both branched and straight-chain saturated aliphatic hydrocarbon groups, having the specified number of carbon atoms. Thus, the term C₁-C₆ alkyl as used herein includes alkyl groups having from 1 to about 6 carbon atoms. When C₀-Cₙ alkyl is used herein in conjunction with another group, for example, phenylC₀-C₄ alkyl, the indicated group, in this case phenyl, is either directly bound by a single covalent bond (C₀), or attached by an alkyl chain having the specified number of carbon atoms, in this case from 1 to about 2 carbon atoms. Examples of alkyl include, but are not limited to, methyl, ethyl, n-propyl, isopropyl, n-butyl, t-butyl, n-pentyl, and sec-pentyl.

“Alkenyl” as used herein, indicates hydrocarbon chains of either a straight or branched configuration comprising one or more unsaturated carbon-carbon bonds, which may occur in any stable point along the chain, such as ethenyl and propenyl.

“Alkynyl” as used herein, indicates hydrocarbon chains of either a straight or branched configuration comprising one or more triple carbon-carbon bonds that may occur in any stable point along the chain, such as ethynyl and propynyl.

“Alkoxy” represents an alkyl group as defined above with the indicated number of carbon atoms attached through an oxygen bridge. Examples of alkoxy include, but are not limited to, methoxy, ethoxy, n-propoxy, propoxy, n-butoxy,2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

As used herein, the term “aryl” indicates aromatic groups containing only carbon in the aromatic ring or rings. Such aromatic groups may be further substituted with carbon or non-carbon atoms or groups. Typical aryl groups contain 1 to 3 separate, fused, or pendant rings and from 6 to about 18 ring atoms, without heteroatoms as ring members. Where indicated aryl groups may be substituted. Such substitution may include fusion to a 5 to 7-membered saturated cyclic group that optionally contains 1 or 2 heteroatoms independently chosen from N, O, and S, to form, for example, a 3,4-methylenedioxy-phenyl group. Aryl groups include, for example, phenyl, naphthyl, including 1-naphthyl and 2-naphthyl, and bi-phenyl.

“Cycloalkyl” as used herein, indicates saturated hydrocarbon ring groups, having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norbornane or adamantane.

“Haloalkyl” indicates both branched and straight-chain saturated aliphatic hydrocarbon groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge.

“Halo” or “halogen” as used herein refers to fluoro, chloro, bromo, or iodo.

As used herein, “heteroaryl” indicates a stable 5- to 7-membered monocyclic or 7-to 10-membered bicyclic heterocyclic ring which contains at least 1 aromatic ring that contains from 1 to 4, or specifically from 1 to 3, heteroatoms chosen from N, O, and S, with remaining ring atoms being carbon. When the total number of S and O atoms in the heteroaryl group exceeds 1, these heteroatoms are not adjacent to one another. In an embodiment, the total number of S and O atoms in the heteroaryl group is not more than 2. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, and 5,6,7,8-tetrahydroisoquinoline. In the term “heteroarylalkyl,” heteroaryl and alkyl are as defined above, and the point of attachment is on the alkyl group. This term encompasses, but is not limited to, pyridylmethyl, thiophenylmethyl, and pyrrolyl(1-ethyl).

The term “heterocyclyl” is used to indicate saturated cyclic groups containing from 1 to about 3 heteroatoms chosen from N, O, and S, with remaining ring atoms being carbon. Heterocyclyl groups have from 3 to about 8 ring atoms, and more typically have from 5 to 7 ring atoms. A C₂-C₇ heterocyclyl group contains from 2 to about 7 carbon ring atoms and at least one ring atom chosen from N, O, and S. Examples of heterocyclyl groups include morpholinyl, piperazinyl, piperidinyl, and pyrrolidinyl groups.

“Pharmaceutically acceptable salts” includes derivatives of the disclosed compounds wherein the parent compound is modified by making an acid or base salt thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile can be used, where practicable.

In certain embodiments, the compounds are administered to a patient or subject. A “patient” or “subject”, used equivalently herein, means mammals and non-mammals. “Mammals” means a member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex. A preferred patient is a human patient.

The phrase “effective amount,” as used herein, means an amount of an agent which is sufficient enough to significantly and positively modify symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

The amount of compound effective for any indicated condition will, of course, vary with the individual subject being treated and is ultimately at the discretion of the medical or veterinary practitioner. The factors to be considered include the condition being treated, the route of administration, the nature of the formulation, the subject's body weight, surface area, age and general condition, and the particular compound to be administered. In general, a suitable effective dose is in the range of about 0.1 to about 500 mg/kg body weight per day, preferably in the range of about 5 to about 350 mg/kg per day. The total daily dose may be given as a single dose, multiple doses, e. g., two to six times per day, or by intravenous infusion for a selected duration. Dosages above or below the range cited above may be administered to the individual patient if desired and necessary.

The RICTOR inhibitors (or inhibitors targeting the RICTOR/mTOR signaling) and EGFR inhibitors can be in the form of pharmaceutical compositions. As used herein, “pharmaceutical composition” means therapeutically effective amounts of the compound together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The invention is further illustrated by the following non-limiting examples.

### EXAMPLES

### Methods

Cell lines and Material: The HCC827 NSCLC cell line was obtained from the American Type Tissue Collection. PC9 cells were a gift from Dr. Susumu Kobayashi (Harvard Medical School, Boston, Mass.). RICTOR amplified HCC827 cells were developed by transfecting HCC827 cells with a doxycycline-inducible plasmid for the overexpression of wild-type RICTOR in the presence of doxycycline (RICTOR-pTetOne, modified from Tet-One Systems, Clontech Laboratories, Inc., and generated by GENEWIZ, Inc). Erlotinib-resistant HCC827 cells were established by methods known in the art. Cells were grown in RPMI 1640 supplemented with 10% FBS and 1× Antibiotics/Antimycotic (Invitrogen). Erlotinib, afatinib, osimertinib and sapanisertib were obtained from Selleck Chemicals (Houston, Tex., USA). All drugs were dissolved in DMSO at 10 mM and stored at −20° C. The final diluted DMSO concentration in all experiments was <0.1% in medium. Cycloheximide was purchased from Sigma-Aldrich (MO, USA)

Immunoblotting and Antibodies: All antibodies were purchased from Cell Signaling Technology (Boston, Mass.). Cell lysate was harvested using RIPA buffer and the supernatant was subjected for following steps after centrifugation. Proteins were subjected to 10% SDS-PAGE gel and transferred onto 0.45 μM nitrocellulose membrane (Biorad, Calif., USA) for western blot analysis. After incubation with primary antibody overnight at 4° C., the membrane was washed and incubated with secondary antibodies. The final blots were developed with a chemiluminesence system (Promega, Wis., USA).

Cell Growth and Viability Assay: The MTS proliferation assay was performed as described in manufacture protocol (Promega, Wis., USA). Cells were seeded at a density of 2000 cells per well in 96-well plates. After incubating with drugs for 72 hours, MTS reagent was added and the absorbance was measured at 490 nm. Viable cell numbers were determined using the MTS assay kit according to the manufacturer's protocol.

Clonogenic Survival Assay: Logarithmically growing cells were plated in triplicate. All drugs were added for 72 hours and withdrawn afterwards. After 10-14 days, colonies were fixed with 70% EtOH and stained with 0.5% crystal violet (Sigma Aldrich, St Louis, Mo.). Surviving colonies were defined as colonies containing >50 cells.

In vitro Cell Migration and Invasion Assay: The transwell assay was performed according to manufacturer's protocol (Corning, N.Y.). Briefly, the cells were starved for 24 hours and then seeded on the transwell insert (Corning, N.Y.) in 12-well plates. The bottom of the well contained medium with 10% FBS. After 24 hours, the bottom of the inserts were fixed with 70% ethanol and stained with 0.5% crystal violet. The cells were counted triplicate under microscope.

In the invasion assay, matrigel (0.8 mg/ml) layer was added on the transwell insert first. After the matrigel layer was solidified, the experiment was performed in the same way as the migration assay.

Inducible shRNA Knockdown: The cells were transfected with the Thermo Scientific Open Biosystems Expression Arrest TRIPZ™ Lentiviral shRNAmir. The transduction was performed per the manufacturer's manual (TRIPZ Human RICTOR shRNA, Thermo Fisher Scientific, Waltham, Mass.).

Xenograft Mouse Studies: Athymic mice (Envigo Laboratory, male, 6-8 weeks old) were inoculated subcutaneously with 5×10⁶ NSCLC cells. After 2 days, mice were divided into a vehicle control (water) group and a doxycycline group, with 6 mice in each group. Doxycycline was administered by oral gavage: 100 mg/kg/day. The same volume of water was given to the control group. A total of 20 daily doses were administered in 4 weeks. Once the tumor reached 50 mm³, erlotinib was administered by oral gavage daily. Tumors were measured with calipers twice weekly and the size was calculated as length×width²/2. The protocol was approved by the Institutional Animal Care and Use Committee.

Caspase-3 calorimetric assay: A caspase-3 calorimetric assay was performed according to manufacturer's protocol. (Millipore, Burlington, Mass.). Briefly, the cell lysates were incubated with Caspase-3 substrate at 37° C. for 2 hours. The absorbance was measured at 405 nm. The Caspase-3 activity was determined by comparing to the OD from the standard curve and control.

Protein stability assay: Cells were first seeded with a density of 6×10⁵ in 35 mm dish. Doxycycline was added 4 h after to induce modified RICTOR expression. After incubation of 24 hours, cycloheximide (300 μg/ml) was added into cells. Within a range of different time points (1 h-24 h), cell lysate was collected and prepared for western blot analysis.

Statistical Analysis: The combination effects were evaluated with an MTT assay. CI values was calculated by calsyn. CI values of less than 1, 1 and greater than 1 were taken as synergism, additive effect, and antagonism, respectively. All data were expressed as mean±SD from at least triplicate experiments. Statistical analysis was performed by excel. Differences were considered significant at P<0.05.

### Example 1

### RICTOR Inhibition Restored the EGFR TKI Sensitivity in Resistant EGFR-Mutant NSCLC Cells

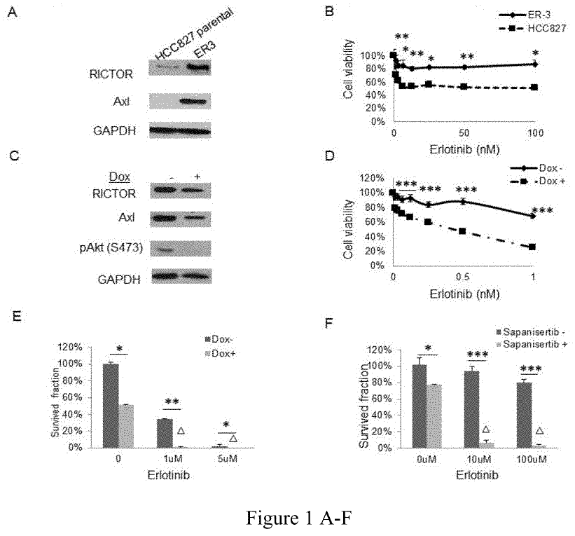

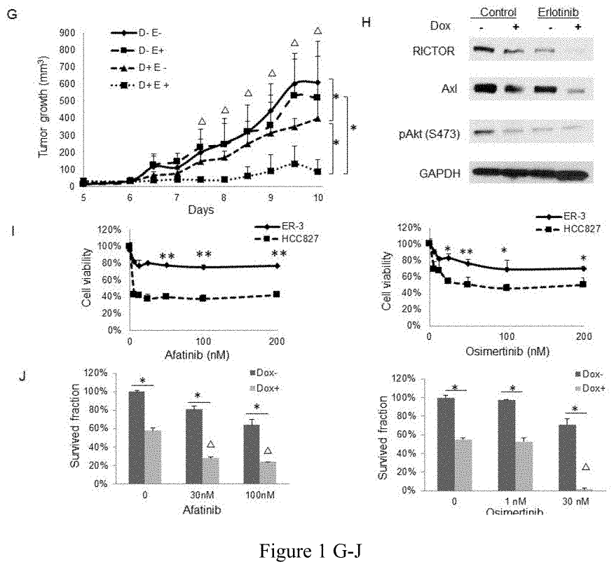

The functional role of RICTOR in mediating acquired EGFR TKI resistance was investigated in both in vitro and in vivo lung cancer models. An erlotinib-resistant HCC827 cell line (ER) was previously developed by the inventors. HCC827 is a NSCLC cell line harboring EGFR exon 19 deletion mutations. ER was developed from an erlotinib-resistant HCC827 clone which is primarily driven by Axl overexpression. The resistance ratio IC_{50(ER)}/IC₅₀₍ₚₐᵣₑₙₜₐₗ₎) comparing ER to parental HCC827 cells was approximately 20000 (FIG. 1B). Axl overexpression was confirmed by western blot analysis. Interestingly, a concurrent and significant RICTOR upregulation was observed in ER cells. (FIG. 1A) To determine the impact of RICTOR blockade on the erlotinib sensitivity, doxycycline-inducible RICTOR knockdown ER cells were generated. RICTOR knockdown markedly improved erlotinib sensitivity, decreasing the IC₅₀ of erlotinib around 1000 fold in ER cells. (FIGS. 1D and 1E) Remarkably, inducible RICTOR knockdown downregulated Axl expression (FIG. 1C). Therefore, the restored erlotinib sensitivity may be due to the downregulated Axl signaling since ER is an Axl overexpression-driven resistant cell model. In addition to genetic knockdown, similar results were also observed with mTORC1/2 pharmacological inhibitor, sapanisertib (TAK228). (FIG. 1F) Hence, RICTOR inhibition effectively restored the erlotinib sensitivity in lung cancer cells in vitro.

The in vivo mouse tumor xenograft model was performed using ER cells. The mice were separated into four groups: with or without inducible RICTOR knockdown (by adding doxycycline) and with or without erlotinib treatment. As expected, with four weeks of treatment, erlotinib alone failed to inhibit tumor growth in this erlotinib-resistant model. On the other hand, RICTOR knockdown alone significantly inhibited the tumor growth, whereas the combined treatment (RICTOR knockdown and erlotinib) markedly blocked tumor growth than either approach alone (P<0.05). (FIG. 1G) Moreover, signaling pathway analysis with the xenograft tumors revealed that the combination treatment (RICTOR knockdown and erlotinib) led to the lowest expression of Axl, concurring with its pronounced therapeutic effects. (FIG. 1H) Therefore, it seems that RICTOR inhibition can restore the erlotinib sensitivity by downregulating Axl expression.

Several recent studies have suggested that Axl overexpression may also be associated with treatment resistance to the second and third generations of EGFR TKIs. Therefore, the drug response of ER was further tested with afatinib (2^{nd} generation) and osimertinib (3^{rd} generation). Consistent with previous reports, Axl-overexpressed ER was associated with resistance to both inhibitors. (FIG. 1I and 1J) By genetically knocking down RICTOR, both sensitivity and IC₅₀ of afatinib and osimertinib were improved. Taken together, it seems that all three generations of EGFR TKIs share common resistant mechanisms involving RICTOR and Axl upregulation.

### Example 2

### RICTOR Upregulation Led to EGFR TKI Resistance

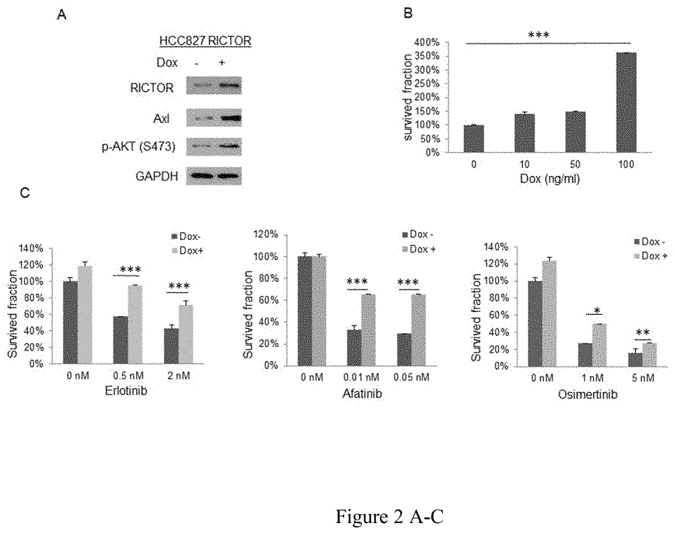

Next, it was determined whether RICTOR upregulation could result in EGFR TKI resistance. A doxycycline-inducible RICTOR upregulation model in HCC827 cells (HCC827 OE) was generated. As anticipated, lung cancer cell growth was promoted by RICTOR upregulation with addition of doxycycline. (FIG. 2B) This confirmed the potential oncogenic function of RICTOR in lung cancer cells. Interestingly, RICTOR upregulation led to elevated Axl expression (FIG. 2A) which is a known resistance mechanism to EGFR TKI. As shown in FIG. 2C, comparing to parental HCC827 cells, the RICTOR-upregulated HCC827 cells were more resistant to erlotinib, afatinib and osimertinb. Therefore, RICTOR upregulation can promote the resistance to all three generations of EGFR TKIs.

### Example 3

### RICTOR Inhibition Further Enhanced the Sensitivity of EGFR TKIs in Sensitive EGFR-Mutant NSCLC Cells

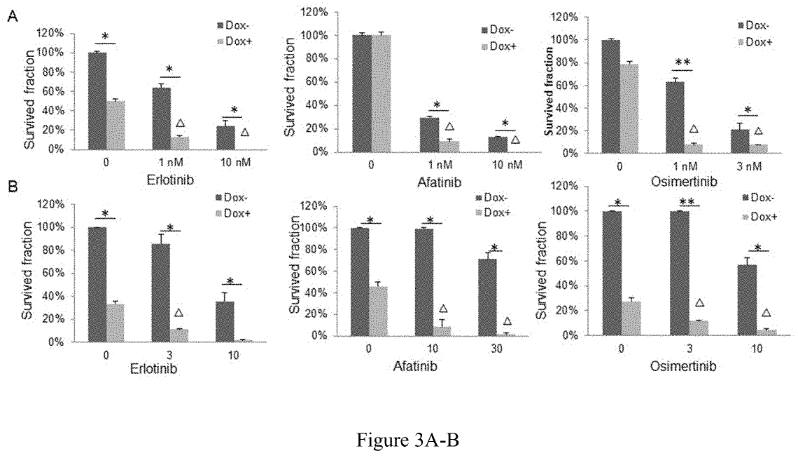

Next it was investigated whether RICTOR inhibition would further improve the EGFR TKI activity in sensitive EGFR-mutant NSCLC cells. The drug response of erlotinib, afatinib and osimertinib was tested with two doxycycline-inducible RICTOR knockdown cell models, HCC827-R4 and PC9-R4. Even though cells responded differently with the three drugs, all of the drugs demonstrated higher inhibitory effects on cell proliferation in RICTOR knockdown cells. The most significant change of IC₅₀ was a decrease of >10 fold of osimertinib in HCC827 cells, and of afatinib in PC-9 cells. (FIGS. 3A and 3B) The cell growth assay was repeated with PC9 parental cells treated with a combined treatment of sapanisertib and EGFR TKIs. FIG. 3C showed that sapanisertib also significantly improved the sensitivity of all three inhibitors in the PC9 cells. Taken together, RICTOR inhibition significantly improved the TKI responses in EGFR sensitive NSCLC cells in vitro.

Mouse tumor xenograft models with HCC827-R4 cells were then examined. As shown in FIG. 3D, erlotinib alone effectively inhibited tumor growth. A further significant reduction in tumor growth was identified with combined erlotinib treatment and RICTOR knockdown (with addition of doxycycline). Taken together, RICTOR inhibition can further increase the efficacy of EGFR TKI in lung cancer.

### Example 4

### RICTOR Mediated EGFR TKI Resistance Through Upregulating Axl Expression

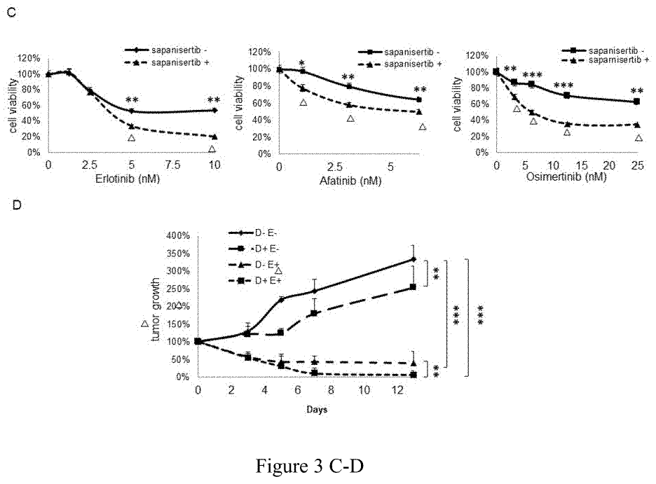

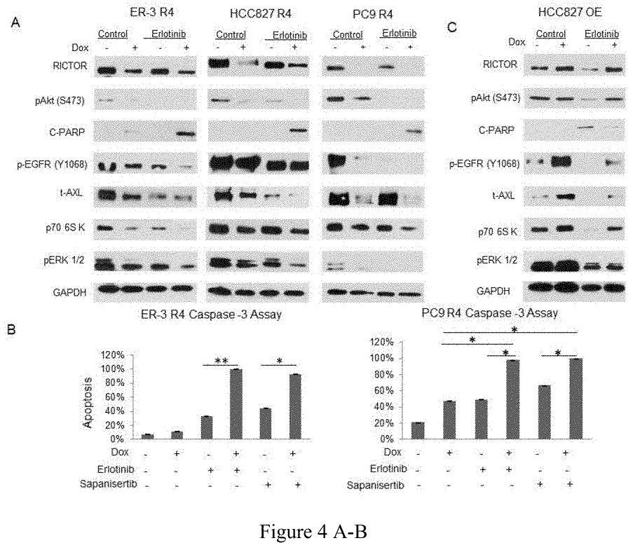

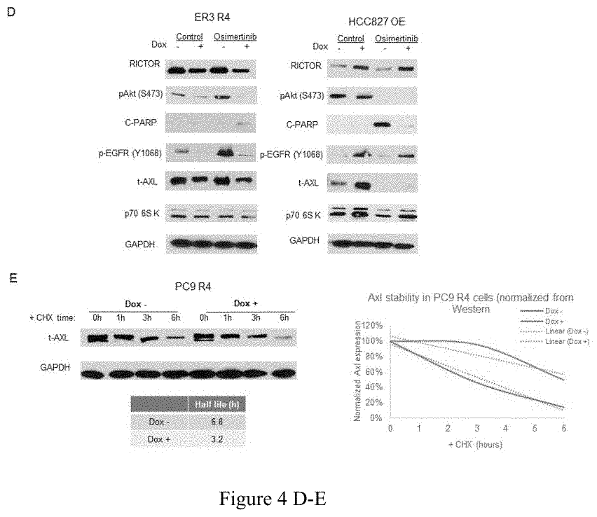

The molecular mechanism of how RICTOR contributed to EGFR TKI resistance was investigated. As aforementioned, RICTOR knockdown led to both reduced Axl expression and improved EGFR TKI sensitivity, indicating that the Axl signaling pathway is a potential mediator involved in the RICTOR-induced EGFR TKI resistance. As shown in FIG. 4A, RICTOR knockdown led to decreased phosphorylation of AKT 5473 in ER-R4, HCC827-R4 and PC9-R4 cells, indicating its on-target inhibition of the RICTOR/mTORC2 activity. RICTOR ablation also resulted in decreased Axl expression and reduced ERK signaling, along with increased apoptosis (as evidenced by elevated c-PARP and caspase-3) in both EGFR resistant ER-3 and sensitive HCC827/PC9 cells. On the other hand, in RICTOR-overexpressed HCC827 OE cells, RICTOR upregulation led to Axl overexpression and decreased apoptosis (as indicated by less c-PARP level). Taken together, activation of the RICTOR signaling is associated with resistance to EGFR-targeted therapy, likely through modulation of the AXL/ERK signaling pathway.

### Example 5

### RICTOR-Induced Axl Upregulation was a Common Resistance Mechanism for EGFR TKI Treatment

To evaluate whether RICTOR-induced Axl upregulation was a common resistant mechanism for EGFR TKIs, Axl signaling in osimertinib-treated ER3-R4 and HCC827 OE cells was examined. As shown in FIG. 4D, RICTOR knockdown led to Axl downregulation, which was associated with more pronounced apoptosis (as evidenced by elevated c-PARP) in osimertinib-treated cells. At the same time, RICTOR upregulation in the HCC827 OE cells resulted in Axl overexpression, and was associated with less apoptosis. Taken together, RICTOR positively regulates EGFR TKI resistance by upregulating Axl expression, which seems to be a common resistance mechanism shared by EGFR TKIs.

### Example 6

### RICTOR Regulated Axl Expression Through Inhibiting Axl Degradation

There had been no reports suggesting the regulatory role of RICTOR on Axl. On the other hand, mTOR inhibition could modulate overall protein degradation and affect cell activity. Thus, protein stability assays were performed to investigate the impact of RICTOR on the half-life of Axl. The inducible RICTOR knockdown PC9 cells (PC9-R4) were treated with cyclohyximide (CHX), a protein synthesis inhibitor, and the turnover rate of Axl expression were calculated. As shown in FIG. 4E, RICTOR knockdown (with addition of doxycycline) led to more rapid Axl degradation. The half-life of Axl was decreased from 6.8 to 3.2 hours upon RICTOR ablation. Taken together, the data suggest that RICTOR knockdown promoted Axl degradation, which subsequently led to Axl downregulation and further impacted it downstream functions, including signaling pathways influencing EGFR therapies. Hence, RICTOR regulates the Axl expression by inhibiting its degradation.

### Example 7

### RICTOR Regulated EMT, Invasion and Migration

RICTOR has been shown to promote tumor metastasis through Epithelial-mesenchymal transition (EMT) in glioblastoma and breast cancer. EMT is a critical step in tumorigenesis and metastasis. EMT is also a known mechanism of resistance to EGFR TKIs. RICTOR expression was positively correlated with N-cadherin (mesenchymal marker) and negatively correlated with E-cadherin expression (epithelial marker), indicating its role on promoting EMT process. (FIG. 5A) Thus, RICTOR-mediated TKI resistance may also occur through EMT. To further investigate this hypothesis, cell migration and invasion were examined since EMT is the essential step involved in these metastatic processes. As expected, in HCC827-R4 and PC9-R4 cells, RICTOR ablation either genetically or pharmacologically led to diminished cell migration and invasion. (FIG. 5B-D) At the same time, in HCC827 OE cells, RICTOR upregulation (by adding doxycycline) promoted cell migration and invasion, which was inhibited by mTOR1/2 inhibitor, sapanisertib. Taken together, activation of RICTOR signaling modulates EMT, which may subsequently contributes to its resistance to EGFR TKIs.

### Conclusions/Discussion Examples 1-7 RICTOR and Resistance to EGFR-Targeted Therapy

The inventors recently reported that a novel subgroup of lung cancer may be defined by the presence of RICTOR amplification. Among them, 27% of had concomitant EGFR alterations. Here, the possible interplay between the two signaling networks and the potential impact of the RICTOR signaling on the efficacy of EGFR-targeted therapy was investigated. Activation of RICTOR signaling could lead to resistance to EGFR-targeted therapy, whereas RICTOR inhibition could significantly improve the sensitivity of all three generations of EGFR TKIs (erlotinib, afatinib and osimertib). This study implicates that RICTOR activation may be a shared resistance mechanism among EGFR TKIs. The combination strategy of RICTOR inhibition and TKIs may be applied to overcome resistance and to improve the efficacy of EGFR-targeted therapy. Moreover, the data suggests that the RICTOR signaling may modulate the resistance to EGFR TKIs through its regulation on Axl degradation and EMT process.

The potential roles of RICTOR amplification on EGFR TKI resistance was first implicated by the index case: RICTOR amplification was acquired after the patient progressed after receiving first generation of TKI treatment. Moreover, this observation is consistent with a recent published NGS analysis: The lung cancer patients with TKI resistance showed frequent RICTOR alterations (3/8), whereas the patients sensitive to TKI were lack of these changes. Taken together, these clinical observations suggest that RICTOR amplification may be a new molecular predictor of EGFR TKI resistance.

To test this hypothesis, the functional roles of RICTOR on EGFR TKI resistance was established using a previously established Axl overexpression-driven erlotinib-resistant HCC827 cell model (ER-3). The ER-3 cells were resistant not only to erlotinib, but also to the newer generations of TKIs, such as afatinib and osimertinib. Osimertinib was recently approved by FDA as the front-line treatment for EGFR-mutated NSCLC, in addition to its prior approval at the second-line setting against the EGFR T790M resistant mutation. Despite its impressive response and survival, osimertinib-treated patients eventually and inevitably develop acquired resistance. The current data is consistent with a recent study and implicates that Axl overexpression renders resistance to EGFR TKIs, including osimertinib.

Moreover, in comparison to the HCC827 parental cells, elevated RICTOR levels were also detected in the Axl-overexpressed ER-3 cells. Remarkably, in the ER-3 cells, RICTOR knockdown (either genetically or pharmacologically) significantly restored the sensitivity and overcame the resistance to all three inhibitors both in vitro and in vivo (FIG. 1). It was then further investigated how RICTOR ablation or upregulation can modulate the efficacy of EGFR-targeted therapy in EGFR TKI sensitive lung cancer. RICTOR ablation further improved sensitivity of all three EGFR TKIs in the sensitive lung cancer cells. In contrast, RICTOR upregulation in those cells led to resistance against all three EGFR TKIs (FIG. 4). Taken together, this study suggests that RICTOR activation or upregulation conveys resistance to EGFR-targeted therapy.

Experiments were then conducted to exploit the molecular mechanisms underlying RICTOR-mediated EGFR TKI resistance. Intriguingly, analyses from both cell lines and xenograft mouse models revealed a positive regulatory role of RICTOR on Axl expression: RICTOR inhibition led to decreased Axl expression, whereas RICTOR upregulation resulted in elevated Axl expression. Both corroborated with their effects on the sensitivity of EGFR TKIs. Axl overexpression is a known resistance mechanisms to EGFR-targeted therapy. Thus, RICTOR signaling may mediate the efficacy of EGFR-targeted therapy through its regulatory effects on Axl expression.

Axl overexpression due to extended degradation rate was recently identified as an osimertinib-resistant mechanism. Targeting Axl degradation could be an effective approach to overcome Axl-related drug resistance. RICTOR has been suggested to regulate protein degradation by affecting ubiquitination and autophagy-mediated degradation process. Therefore, the impact of RICTOR on the protein turnover rate of Axl was examined. RICTOR knockdown markedly shortened the half-life of Axl. Taken together, it seems that RICTOR can modulate Axl expression by negatively regulating its degradation, which may subsequently contribute to EGFR TKI resistance.

EMT is a hallmark of tumor metastasis, which represents a cell morphological change to acquire a spindle-like fibroblastic phenotype. EMT is also an established resistance mechanisms to EGFR TKIs. Thus, in the current study, it was determined whether RICTOR could modulate EMT process. RICTOR upregulation promoted EMT in HCC827 RICTOR cells, whereas RICTOR inhibition resulted in a reversed EMT process, named MET, in the ER-3 R4 cells. This observation is consistent with previous report suggesting the potential roles of RICTOR on EMT. Thus, upregulated EMT may be also involved in RICTOR-mediated EGFR TKI resistance.

In summary, these results indicate that RICTOR activation is associated with resistance to EGFR-targeted therapy, likely through its regulation on Axl degradation and EMT process. This study is also the first suggesting the regulatory role of RICTOR on Axl expression. Moreover, effective ablation of the RICTOR signaling pathway can restore the sensitivity of EGFR TKIs, and may represent a novel strategy to overcome resistance to molecularly targeted therapy in subsets of NSCLC patients.

### Example 8

### Genetic Charaterization of Metastases (Particularly Brain Metasteses) in Lung Cancer, Focuing on the PI2K/AKT/AKT/mTOR Pathway

The metastatic cascade is a complex process involving multiple steps. A variety of signaling pathways play important roles, such as EGFR/RAS/RAF/MEK/ERK, HGF/MET, and PI3K/AKT/mTOR signaling. As aforementioned, prior studies with limited cases comparing primary tumor to distant metastasis suggested branched evolution and genomic heterogeneity. These studies (<100 cases per study) further implicated a potential association of PI3K/AKT/mTOR pathway with the development of brain metastases. Little is known about the genetic signatures associated with particular metastatic sites in lung cancer.

To better understand the underlying biology of metastasis, 11845 cases of lung adenocarcinoma (including 8412 primary lung tumors, 843 brain metastases, and 2590 other metastatic sites) that underwent comprehensive genomic profiling test (FoundationOne®) were reviewed (data not shown).

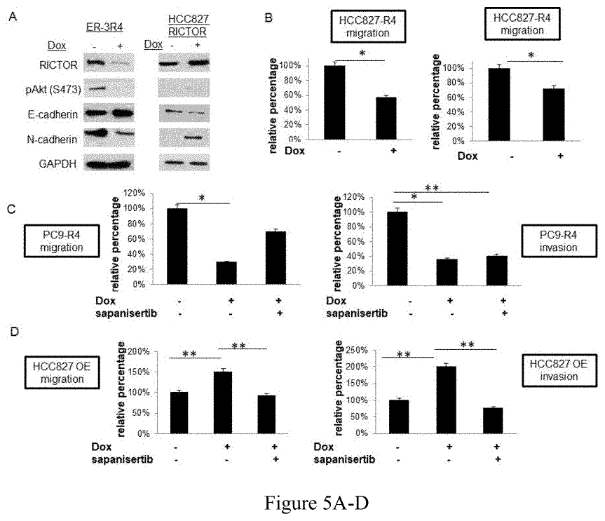

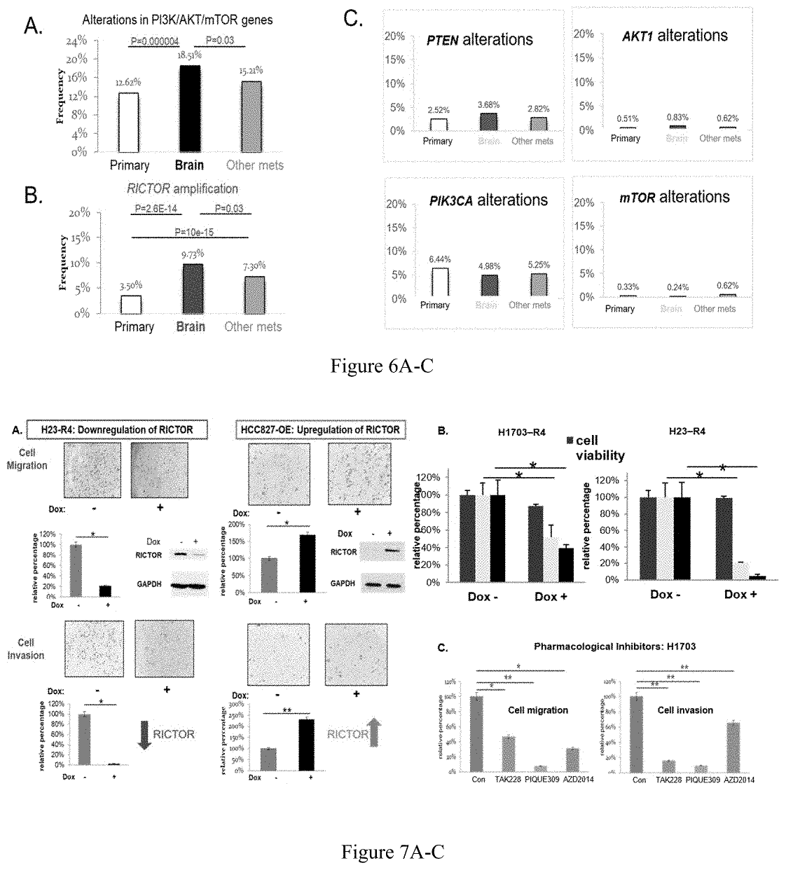

Compared to primary tumor, significantly more frequent PI3K/AKT/mTOR gene alterations in the metastatic sites, especially brain metastases (brain vs. primary: 18.5% vs. 12.6%, P=0.000004; other mets vs. primary: 15.2% vs. 12.6%, P=0.04; brain vs. other mets: 18.5% vs. 15.2%, P=0.03) in lung adenocarcinoma (FIG. 6).

Among all tested genes in this pathway, RICTOR amplification is associated with the highest enrichment in brain metastases (brain vs. primary: 9.73% vs 3.50%, P=2.6E-14; brain vs. other mets: 9.73% vs. 7.3%, P=0.03); There is also significantly more frequent RICTOR amplification in other metastatic sites as compared to primary tumor (other mets vs. primary: 7.3% vs.3.5%, P=10E-15) (FIG. 6).

In contrast, PTEN, AKT1, PIK3CA or mTOR genetic alterations show similar prevalence in the primary tumor, brain and other metastatic sites (FIG. 6C). Thus, it appears that the overall enrichment in the PI3K/AKT/mTOR pathway in the brain metastases, is primarily driven by RICTOR amplification. Similarly, there are trends toward more frequent RICTOR amplification in the brain metastases in other lung cancer histological subgroups (such as 2553 cases of squamous cell lung cancer).

Similarly, there are trends toward more frequent RICTOR amplification in the brain metastases in other lung cancer histological subgroups (such as 2553 cases of squamous cell lung cancer). It appears that the overall enrichment in the PI3K/AKT/mTOR pathway in the metastatic sites (especially brain metastases), is primarily driven by RICTOR amplification.

### Example 9

### RICTOR Modulates Migration and Invastion In Vitro

Without being held to theory, it was hypothesized that RICTOR amplification is important to drive the pathogenesis of metastasis. A series of lung cancer cell lines with inducible RICTOR downregulation or upregulation was previously designed. Migration and invasion are key steps involved in metastasis. As shown in FIG. 7A and B, inducible RICTOR knockdown in H23 lung cancer cells reduces cell migration and invasion, whereas upregulation of RICTOR in HCC827 lung cancer cells promotes these processes. The parental H23 cell line is RICTOR-amplified, while the parental HCC827 cells have two copies of the RICTOR gene.

Several lines of evidence suggest that patients with RICTOR amplification may benefit from treatment with mTOR1/2 inhibitors. The effects of mTOR1/2 inhibitors with known CNS penetrance given the intriguing findings in brain metastasis were studied, including TAK228 (sapanisertib), PQR309 and AZD2014 (vistusertib). All of them have been used in clinical trials. As shown in FIG. 7C, these mTOR inhibitors significantly reduced migration and invasion in vitro in RICTOR-amplified H23 and H1703 lung cancer cells.

### Example 10

### RICTOR Ablation Reduces Lund Cancer Brain Metstasis In Vivo

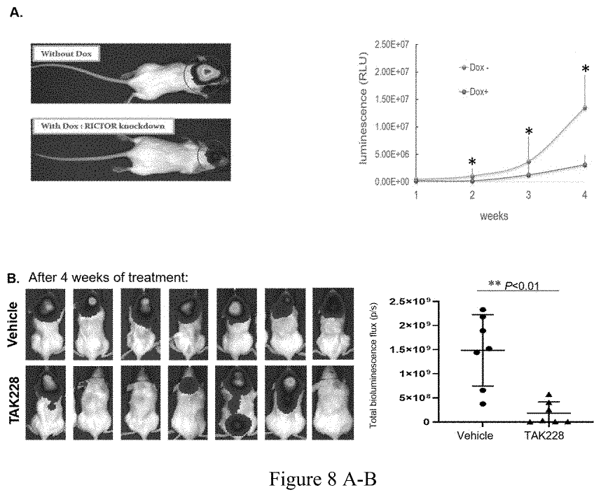

To further test whether RICTOR amplification affects in vivo metastasis, either the inducible H23-R4 or the parental H23 lung cancer cells labeled with luciferase reporter gene (H23-R4-Luc) were implanted in mouse brain stereotactically. Inducible ablation of RICTOR significantly decreased lung cancer tumor growth of H23-R4-Luc in the brain (P<0.05, FIG. 8A). Similarly, TAK228 (Sapanisertib), an mTOR inhibitor as aforementioned, significantly reduced tumor growth in the brain by approximately 75%, including a number of near complete responses (P<0.01, FIG. 8B).

### Example 11

### Molecular Mechanisms Underlying RICTOR-Mediated Metastasis

The invasion-metastatic cascade is driven by multiple signaling pathways and cellular process. One of the primary pathways involved in metastases is epithelial to mesenchymal transition (EMT). Evidence suggests that EMT-associated cellular reprogramming is essential for the formation of metastasis. Moreover, the CXCL12 chemokine-CXCR4 axis plays critical roles in metastasis, especially brain metastases, likely through the establishment of a bridge between cancer cells and the tumor microenvironment. Moreover, to identify potential downstream effectors, we further performed whole-transcriptome analysis with RNAseq studies.

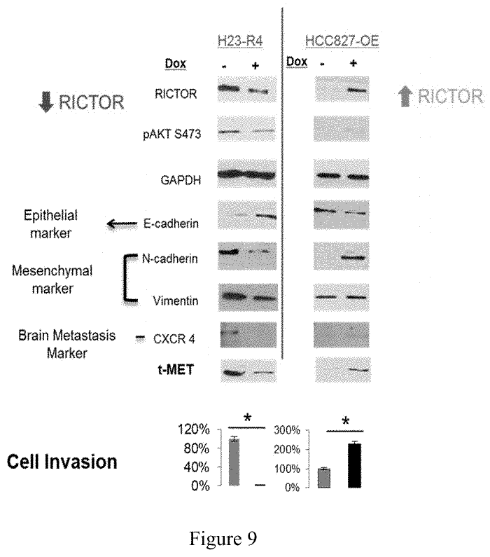

As shown in FIG. 9, RICTOR ablation in H23 cells promoted the expression of E-cadherin (epithelial marker), suppresseed the expression of N-cadherin and vimentin (mesenchymal markers), as well as decreased the level of pAKT and CXCR4 (a marker of brain metastasis) (FIG. 9). The opposite effects on these markers are noted with RICTOR upregulation in HCC827 cells (FIG. 9). Therefore, the results suggest that RICTOR signaling regulates several key factors known to be involved in metastases, such as AKT, epithelial to mesenchymal transition (EMT), CXCL12 chemokine-CXCR4 axis.

### Example 12

### AKT Signaling Contributes to RICTOR-Mediated Metastasis

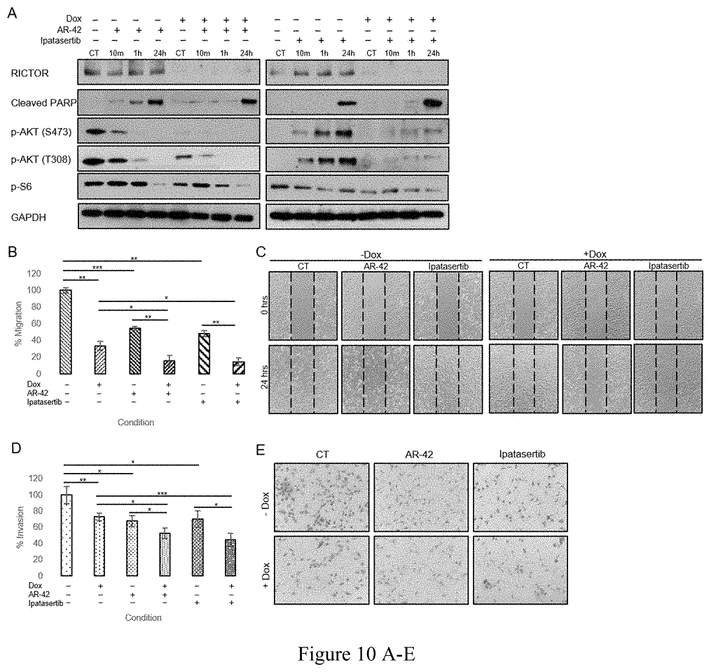

AKT is a downstream effector of the RICTOR-mTOCR2 pathway, to determine its role in RICTOR-mediated metastasis, the effects of two structurally different AKT inhibitors, AR-42 and ipatasertib were inestigated. Although the 24-hour treatment did not result in significant change in cell proliferation, both AKT inhibitors significantly decreased lung cancer cell invasion and migration, associated with increased apoptosis (as shown by elevated cleaved PARP) (FIG. 10A-E). Moreover, the addition of AKT inhibitors to inducible RICTOR knockdown led to further inhibition of AKT signaling, more pronounced apoptosis, and significantly further reduced invasion and migration.

### Example 13

### CXCR4 is a Novel Downstream Target of RICTOR Signaling

Through interaction with its ligand CXCR12, the chemokine receptor CXCR4 has been shown to activate various oncogenic pathways, and subsequently promote cancer cell proliferation, migration, metastasis (especially brain metastasis), immune evasion and resistance to chemotherapy. As shown in two meta-analyses, CXCR4 expression in non-small cell lung cancer was associated with distant metastasis and shorter survival.

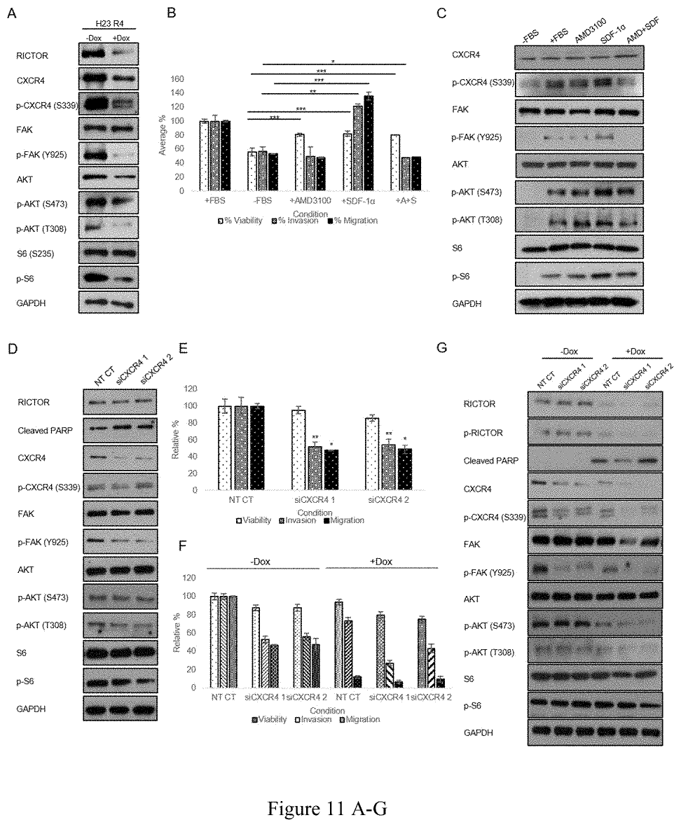

Inducible RICTOR knockdown resulted in less total CXCR4, and inhibited the activation of CXCR4 pathway with reduced p-CXCR4 and p-FAK (FIG. 11A), indicating that RICTOR is an upstream regulator of the CXCR4 pathway.

Activation of the CXCR4 pathway with treatment of SDF-1α led to increased p-FAK, (p-AKT), and p-S6, along with increased migration and invasion. On the other hand, AMD3100, a pharmacological CXCR4 inhibitor, significantly reduced SDF-1α-induced invasion and migration, through blocking CXCR4/FAK/S6 signaling. (FIG. 11B and C). CXCR4 signaling was further blocked with two different CXCR4 siRNAs, which did not affect the total levels of RICTOR, FAK, AKT or S6, or p-AKT; but CXCR4 knockdown significantly reduced the expression of CXCR4, along with p-CXCR4, p-FAK and p-S6. The CXCR4 ablation also significantly reduced lung cancer cell migration and invasion. Moreover, the addition of CXCR4 siRNAs to inducible RICTOR knockdown led to further inhibition of CXCR4 signaling, more pronounced apoptosis, and significantly further reduced invasion and migration (FIG. 11D to G).

### Example 14

### RICTOR Knockdown Significantly Inhibited Lung Cancer Brain Metastasis in the In Vitro Metastasis Assays Using the Stereotactic Brain Injection Models

RICTOR knockdown both genetically (with inducible blockade by adding doxycycline, FIG. 2A) and pharmacologically (with TAK228, FIG. 2B) remarkably reduced the lung cancer growth in the brain.

### Conclusions/Discussion Examples 8-14

### RICTOR and Brain Metastasis

Based on the analysis using an extensive database with 11845 cases of lung adenocarcinoma, it was found that there are more frequent PI3K/AKT/mTOR gene alterations in the metastatic sites, especially brain metastases. Among them, the most prevalent alteration in the metastatic sites, particularly brain metastasis, is RICTOR amplification (FIG. 1). For the first time, an actionable target particularly enriched in brain metastases has been identified.

The in vitro preliminary results further revealed that RICTOR knockdown (either genetically or pharmacologically with mTOR1/2 inhibitors) led to decreased migration and invasion, the key steps involved in metastasis. Moreover, the in vivo mouse models (FIG. 2) showed that RICTOR knockdown (either genetically or pharmacologically with TAK228) significantly reduced tumor growth in the brain. These results imply that activation of RICTOR signaling promotes brain metastasis, whereas blockade of the RICTOR pathway (either genetically or with mTOR1/2 inhibitor) inhibits brain metastasis. Taken together, the data suggests that RICTOR amplification may represent a novel therapeutic target in the treatment of lung cancer metastasis, especially brain metastases. These findings further implicate that inhibitors targeting RICTOR amplification or RICTOR pathways will likely play key roles for the treatment and even prevention of relapse/metastases in lung cancer.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating or preventing metastases in a patient with cancer and at risk for metastases, exhibiting symptoms of metastases, or identified with metastases, comprising administering a therapeutically effective amount of a RICTOR inhibitor for the treatment of metastases, wherein the RICTOR inhibitor is selected from
a) a compound having the structure of formula I or a pharmaceutically acceptable salt thereof: 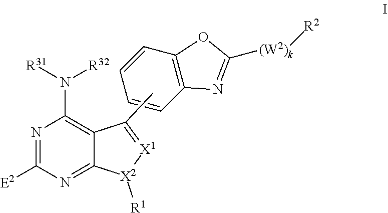
wherein:
X¹ is N or C-E¹ and X² is N; or X¹ is NH or CH-E¹ and X² is C;
R¹ is hydrogen, -L-C₁-C₁₂alkyl, -L-C₃-C₈cycloalkyl, -L-C₁-C₁₂alkyl-C₃-C₈cycloalkyl, -L-aryl, -L-heteroaryl, -L-heterocyclyl, -L-C₁-C₁₂alkylaryl, -L-C₁-C₁₂alkylheterocyclyl, -L-C₁-C₁₂alkylheterocyclyl, wherein each R¹ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
L is absent, —(C═O)—, —C(═O)O—, —C(═O)N(R³¹)—, —S—, —S(═O)—, —S(═O)₂—, —S(═O)₂N(R³¹)—, or —N(R³¹);
k is 0 or 1;
E¹ and E² are independently —(W¹)ⱼ—R³⁴;
j in E¹ or j in E², is independently 0 or 1;
W¹ is —O—, —NR⁷—, —S(═O)₀₋₂—, —C(═O)—, —C(═O)N(R⁷)—, —N(R⁷)C(═O)—, —N(R⁷)S(═O)—, —N(R⁷)S(═O)₂—, —C(══O)O—, —CH(R⁷)N(C(═O)OR⁸)—, —CH(R⁷)N(C(═O)R⁸)—, —CH(R⁷)NS(═O)₂O—, —CH(R⁷)N(R⁸)—, —CH(R⁷)C(═O)N(R⁸)—, —CH(R⁷)N(R⁸)C(═O)—, —CH(R⁷)N(R⁸)S(═O)—, or —CH(R⁷)N(R⁸)S(═O)₂—;
W² is —O—, —NR⁷, —S(═O)₀₋₂—, —C(═O)—, —C(═O)N(R⁷)—, —N(R⁷)C(═O)—, —N(R⁷)C(═O)N(R⁸)—, —N(R⁷)S(═O)—, —N(R⁷)S(═O)₂—, —C(═O)O—, —CH(R⁷)N(C(═O)OR⁸)—, —CH(R⁷)N(C(═O)R⁸)—, —CH(R⁷)N(SO₂R⁸)—, —CH(R⁷)N(R⁸)—, —CH(R⁷)C(═O)N(R⁸)—, —CH(R⁷)N(R⁸)C(═O)—, —CH(R⁷)N(R⁸)S(═O)—, or —CH(R⁷)N(R⁸)S(═O)₂—;
R² is hydrogen, halogen, —OH, —R³¹, —CF₃, —OCF₃, —OR³¹, —NR³¹R³²,—C(═O)R³¹, —CO₂R³¹, —C(═O)NR³¹R³²,—NO₂, —CN, aryl, heteroaryl, C₁-C₁₂alkyl, C₁-C₁₂haloalkyl, or C₃-C₈cycloalkyl, wherein each of the aryl or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
R³⁴ is hydrogen, halogen, —OH, —R³¹, —CF₃, —OCF₃, —OR³¹, —NR³¹R³², —C(═O)R³¹, —CO₂R³¹, —C(═O)NR³¹R³², —NO₂, —CN, —NR³¹C(═O)R³², —NR³¹C(═O)OR³², aryl, heteroaryl, heterocyclyl, C₁-C₁₂alkyl, C₁-C₁₂haloalkyl, or C₃-C₈cycloalkyl, wherein each of the aryl, heteroaryl, or heterocyclyl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
each R³¹ and R³² independently is hydrogen or C₁-C₁₂alkyl, wherein the C₁-C₁₂alkyl is optionally substituted with aryl, heterocyclyl, or heteroaryl group, wherein each of the aryl, heterocyclyl, or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN; and
each R⁷ and R⁸ independently is hydrogen, C₁-C₁₂alkyl, aryl, heteroaryl, heterocyclyl or C₃-C₁₀cycloalkyl, wherein each R⁷ and R⁸ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
b) a compound having the structure of formula II or a pharmaceutically acceptable salt thereof: 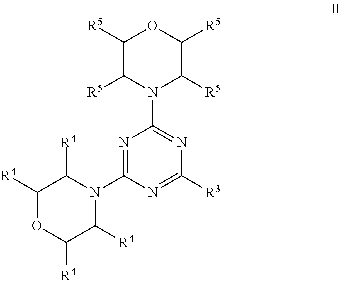
wherein:
R³ is an aryl or heteroaryl group, specifically pyridyl, pyridazine, pyrimidine, or pyrazine, wherein R³ is optionally substituted with 1, 2, or 3 substituents, each substituent independently is halogen, C₁-C₁₂ alkyl, C₁-C₁₂ haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)², —NR¹⁰C(═O)NR¹⁰R¹¹, —C(═O)OR¹⁰, or —C(═O)NR¹⁰R¹¹;
each R⁴ and R⁵ are independently hydrogen, halogen, or C₁-C₁₂ alkyl, specifically hydrogen or C₁-C₂ alkyl;
each instance of R¹⁰ and R¹¹ independently is hydrogen, C₁-C₁₂ alkyl, or C₃-C₁₂ carbocyclyl, or
R¹⁰ and R¹¹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
c) a compound having the structure of formula III or a pharmaceutically acceptable salt thereof: 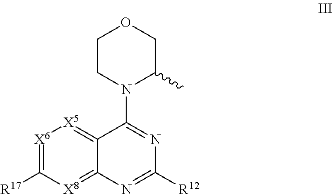
wherein:
one or two of X⁵, X⁶ and X⁸ is N, and the others are CH;
R¹⁷ is hydrogen, halogen, C₁-C₁₂ alkyl, C₁-C₁₂ haloalkyl, OR¹⁰, SR¹⁰, NR¹⁰R¹¹, aryl, or heteroaryl, wherein the aryl and heteroaryl are optionally substituted with 1, 2, or 3 substituents, each substituent independently is halogen, C₁-C₁₂ alkyl, C₁-C₁₂ haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)₂, —NR¹⁰C(═O)NR¹⁰R¹¹, —C(═O)OR¹⁰, or —C(═O)NR¹⁰R¹¹;
each instance of R¹⁰ and R¹¹ independently is hydrogen, C₁-C₁₂ alkyl, or C₃-C₁₂ carbocyclyl, or
R¹⁰ and R¹¹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
R¹² is hydrogen, halogen, C₁-C₁₂ alkyl, C₁-C₁₂ haloalkyl, OR¹⁰, SR¹⁰, NR¹⁸R¹⁹, aryl, or heteroaryl;
R¹⁸ and R¹⁹ are independently hydrogen, C₁-C₁₂ alkyl, or C₃-C₁₂ carbocyclyl, or
R¹⁸ and R¹⁹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, specifically R¹⁸ and R¹⁹ together with the nitrogen to which they are attached form an optionally substituted morpholino; or
d) a compound having the structure of formula IV or a pharmaceutically acceptable salt thereof: 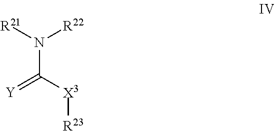
wherein:
R²¹ is aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂, —NR³¹R³², —CO₂R³¹, —CO₂aryl, —C(═O)NR³¹R³², —NO₂, or —CN;
R²² is C₁-C₁₂alkyl, aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
R²³ is C₁-C₁₂alkyl, aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
each R³¹ and R³² independently is hydrogen or C₁-C₁₂alkyl, wherein the C₁-C₁₂alkyl is optionally substituted with aryl, heterocyclyl, or heteroaryl group, wherein each of the aryl, heterocyclyl, or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², −NO₂, or —CN;
X³ is C(R⁴¹R⁵¹), N(R⁴¹), or O, wherein each R⁴¹ and R⁵¹ independently is hydrogen or C₁-C₁₂ alkyl, specifically X³ is N(R⁴¹), more specifically X³ is N(H); and
Y is S or O, specifically O.

2. The method of claim 1, wherein the cancer is lung cancer, breast cancer, colon cancer, kidney cancer, or melanoma.

3. The method of claim 2, wherein the lung cancer is non-small cell lung cancer.

4. The method of claim 3, wherein the lung cancer is advanced stage non-small-cell lung cancer.

5. The method of claim 1, wherein the metastases are brain metastases.

6. The method of claim 1, wherein the metastases are liver, kidney, bone, adrenal, lymph node, or pleural metastases.

7. The method of claim 1, wherein the patient is exhibiting symptoms of brain metastases selected from changes in cognitive ability; headaches; weakness; nausea; vomiting; dizziness; balance issues; difficult speaking; numbness; tingling sensations; behavioral and personality changes; difficulty swallowing; seizures; and combinations thereof.

8. The method of claim 1, further comprising, prior to administering, identifying brain metastases in the patient using magnetic resonance imaging (MRI), computerized tomography (CT), and/or positron emission tomography (PET).

9. A method of reducing resistance to an EGFR, ALK or MET inhibitor in a cancer patient being administered the EGFR, ALK or MET inhibitor, comprising co-administering a therapeutically effective amount of a RICTOR inhibitor and the EGFR, ALK or MET inhibitor, and wherein the RICTOR inhibitor is selected from
a) a compound having the structure of formula I or a pharmaceutically acceptable salt thereof: 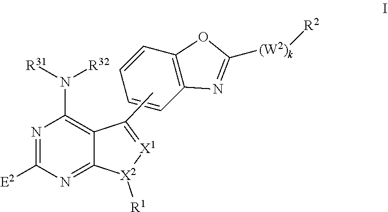
wherein:
X¹ is N or C-E¹ and X² is N; or X¹ is NH or CH-E¹ and X² is C;
R¹ is hydrogen, -L-C₁-C₁₂alkyl, -L-C₃-C₈cycloalkyl, -L-C₁-C₁₂alkyl-C₃-C₈cycloalkyl, -L-aryl, -L-heteroaryl, -L-heterocyclyl, -L-C₁C₁₂alkylaryl, -L-C₁-C₁₂alkylheteroaryl, -L-C₁-C₁₂alkylheterocyclyl, wherein each R¹ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R₃₂, —NO₂, or —CN;
L is absent, —(C═O)—, —C(═O)—, —C(═O)N(R³¹)—, —S—, —S(═O)—, —S(═O)₂—, —S(═O)₂N(R³¹)—, or —N(R³¹);
k is 0 or 1;
E¹ and E² are independently —(W¹)ⱼ—R³⁴;
j in E¹ or j in E², is independently 0 or 1;
W¹ is —O—, —NR⁷, —S(═O)₀₋₂—, —C(═O)—, —C(═O)N(R⁷)—, —N(R⁷)C(═O)—, —N(R⁷)S(═O)—, —N(R⁷)S(═O)₂—, —C(══O)O—, —CH(R⁷)N(C(═O)OR⁸)—, —CH(R⁷)N(C(═O)R⁸)—, —CH(R⁷)NS(═O)₂O—, —CH(R⁷)N(R⁸)—, —CH(R⁷)C(═O)N(R⁸)—, —CH(R⁷)N(R⁸)C(═O)—, —CH(R⁷)N(R⁸)S(═O)—, or —CH(R⁷)N(R⁸)S(═O)₂—;
W² is —O—, —NR⁷, —S(═O)₀₋₂—, —C(═O)—, —C(═O)N(R⁷)—, —N(R⁷)C(═C)—, —N(R⁷)C(═O)N(R⁸)—, —N(R⁷)S(═O)—, —N(R⁷)S(═O)₂—, —C(═O)O—, —CH(R⁷)N(C(═O)OR⁸)—, —CH(R⁷)N(C(═O)R⁸)—, —CH(R⁷)N(SO₂R⁸)—, —CH(R⁷)N(R⁸)—, —CH(R⁷)C(═O)N(R⁸)—, —CH(R⁷)N(R⁸)C(═O)—, —CH(R⁷)N(R⁸)S(═O)—, or —CH(R⁷)N(R⁸)S(═O)₂—;
R² is hydrogen, halogen, —OH, —R³¹, —CF₃, —OCF₃, —OR³¹, —NR³¹R³²,—C(═O)R³¹, —CO₂R³¹, —C(═O)NR³¹R³²,—NO₂, —CN, aryl, heteroaryl, C₁-C₁₂alkyl, C₁-C₁₂ haloalkyl, or C₃-C₈cycloalkyl, wherein each of the aryl or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
R³⁴ is hydrogen, halogen, —OH, —R³¹, —CF₃, —OCF₃, —OR³¹, —NR³¹R³², —C(═O)R³¹, —CO₂R³¹, —C(═O)NR³¹R³², —NO₂, —CN, —NR³¹C(═O)R³², —NR³¹C(═O)OR³², aryl, heteroaryl, heterocyclyl, C₁-C₁₂alkyl, C₁-C₁₂haloalkyl, or C₃-C₈cycloalkyl, wherein each of the aryl, heteroaryl, or heterocyclyl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
each R³¹ and R³² independently is hydrogen or C₁-C₁₂alkyl, wherein the C₁-C₁₂alkyl is optionally substituted with aryl, heterocyclyl, or heteroaryl group, wherein each of the aryl, heterocyclyl, or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO_{2,} or —CN; and
each R⁷ and R⁸ independently is hydrogen, C₁-C₁₂alkyl, aryl, heteroaryl, heterocyclyl or C₃-C₁₀cycloalkyl, wherein each R⁷ and R⁸ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
b) a compound having the structure of formula II or a pharmaceutically acceptable salt thereof: 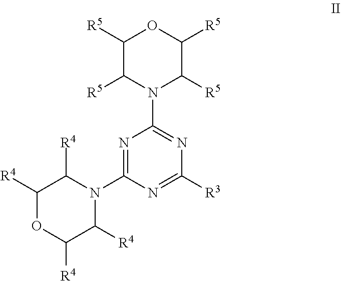
wherein:
—R³ is an aryl or heteroaryl group, specifically pyridyl, pyridazine, pyrimidine, or pyrazine, wherein R³ is optionally substituted with 1, 2, or 3 substituents, each substituent independently is halogen, C₁-C₁₂ alkyl, C₁-C₁₂haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)², —NR¹⁰C(═O)NR¹⁰R¹¹, —C(═O)OR¹⁰, or —C(═O)NR¹⁰R¹¹;
each R⁴ and R⁵ are independently hydrogen, halogen, or C₁-C₁₂ alkyl, specifically hydrogen or C₁-C₂ alkyl;
each instance of R¹⁰ and R¹¹ independently is hydrogen, C₁-C₁₂ alkyl, or C₃-C₁₂ carbocyclyl, or
R¹⁰ and R¹¹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
c) a compound having the structure of formula III or a pharmaceutically acceptable salt thereof: 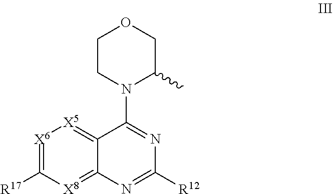
wherein:
one or two of X⁵, X⁶ and X⁸ is N, and the others are CH;
R¹⁷ is hydrogen, halogen, C₁-C₁₂ alkyl, C₁-C₁₂ haloalkyl, OR¹⁰, SR¹⁰, NR¹⁰R¹¹, aryl, or heteroaryl, wherein the aryl and heteroaryl are optionally substituted with 1, 2, or 3 substituents, each substituent independently is halogen, C₁-C₁₂ alkyl, C₁-C₁₂ haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)₂, —NR¹⁰C(═O)NR¹⁰R¹¹, —C(═O)OR¹⁰, or —C(═O)NR¹⁰R¹¹,
each instance of R¹⁰ and R¹¹ independently is hydrogen, C₁-C₁₂ alkyl, or C₃-C₁₂ carbocyclyl, or
R¹⁰ and R¹¹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
R¹² is hydrogen, halogen, C₁-C₁₂ alkyl, C₁-C₁₂ haloalkyl, OR¹⁰, SR¹⁰, NR¹⁸R¹⁹, aryl, or heteroaryl;
R¹⁸ and R¹⁹ are independently hydrogen, C₁-C₁₂ alkyl, or C₃-C₁₂ carbocyclyl, or
R¹⁸ and R¹⁹ together with the nitrogen to which they are attached optionally form a C₃-C₂₀ heterocyclic ring optionally containing one or more additional ring atoms N, O or S, wherein the heterocyclic ring is optionally substituted with 1, 2, or 3 substituents, each substituent independently is oxo, halogen, C₁-C₁₂ alkyl, C₁-C₁₂ haloalkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, specifically R¹⁸ and R¹⁹ together with the nitrogen to which they are attached form an optionally substituted morpholino; or
d) a compound having the structure of formula IV or a pharmaceutically acceptable salt thereof: 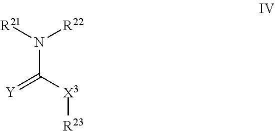
wherein:
R²¹ is aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂R³¹, —CO₂aryl, —C(═O)NR³¹R³², —NO₂, or —CN;
R²² is C₁-C₁₂alkyl, aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
R²³ is C₁-C₁₂alkyl, aryl, heteroaryl, or heterocyclyl, each of which is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
each R³¹ and R³² independently is hydrogen or C₁-C₁₂alkyl, wherein the C₁-C₁₂alkyl is optionally substituted with aryl, heterocyclyl, or heteroaryl group, wherein each of the aryl, heterocyclyl, or heteroaryl group is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R₃₂, —NO₂, or —CN;
X³ is C(R⁴¹R⁵¹), N(R⁴¹), or O, wherein each R⁴¹ and R⁵¹ independently is hydrogen or C₁-C₁₂ alkyl, specifically X³ is N(R⁴¹), more specifically X³ is N(H); and
Y is S or O, specifically O.

10. The method of claim, 9 wherein the inhibitor is an EGFR inhibitor and the patient exhibits an EGFR mutation.

11. The method of claim 10, wherein the EFFR mutation is a T790M secondary mutation in exon 20, MET amplification, hepatocyte growth factor (HGF) overexpression and/or a PIK3CA mutation.

12. The method of claim 9, wherein the EGFR inhibitor is a first generation EGFR inhibitor selected from gefitinib, icotinib and erlotinib.

13. The method of claim 9, wherein the EGFR inhibitor is a second generation EGFR inhibitor selected from afatinib, and dacomitinib.

14. The method of claim 9, wherein the EGFR inhibitor is a third generation EGFR inhibitor selected from osimertinib, olmutinib, PF-06747775, YH5448, avitinib and rociletinib.

15. The method of claim 9, wherein the MET inhibitor is capmatinib, tepotinib, crizotinib, cabozantinib, foretinib, tibantinib, or savolitinib.

16. The method of claim 9, wherein the ALK inhibitor is crizotinib, alectinib, brigatinib, lorlatinib or ceritinib.

17. The method of claim 1, wherein the RICTOR inhibitor is of Formula I, and
X¹ is N and X² is N;
R¹ is hydrogen, -L-C₁-C₁₂alkyl, -L-C₃-C₈cycloalkyl, or -L-aryl, wherein each R¹ that is not hydrogen is optionally substituted with 1, 2, or 3 substituents, each substituent independently is a C₁-C₁₂ alkyl, C₁-C₁₂ haloalkyl, halogen, —OR³¹, —SH, NH₂,—NR³¹R³², —CO₂R³¹, —CO₂aryl, —C(═O)NR³¹R³², —NO₂, or —CN;
L is absent;
k is 0;
E² is —(W¹)ⱼ—R³⁴;
j is 0;
R² is hydrogen, halogen, —OH, —R³¹, —CF₃, —OCF₃, —OR³¹, —NR³¹R³²,—C(═O)R³¹, —CO₂R³¹, —C(═O)NR³¹R³²,—NO₂, —CN, aryl, heteroaryl, C₁-C₁₂alkyl, C₁-C₁₂haloalkyl, or C₃-C₈cycloalkyl;
R³⁴ is hydrogen, halogen, —OH, —R³¹, —CF₃, —OCF₃, —OR³¹, —NR³¹R³², —C(═O)R³¹, —CO₂R³¹, —C(═O)NR³¹R³², —NO₂, —CN, aryl, heteroaryl, heterocyclyl, C₁-C₁₂alkyl, C₁-C₁₂haloalkyl, or C₃-C₈cycloalkyl; and
each R³¹ and R³² independently is hydrogen or C₁-C₁₂alkyl.

18. The method of claim 1, wherein the RICTOR inhibitor is of Formula I, and is sapanisertib.

19. The method of claim 1, wherein the RICTOR inhibitor is of Formula II, and
R³ is pyridyl, pyridazine, pyrimidine, or pyrazine, where R³ is optionally substituted with 1 or 2 substituents, each substituent independently is halogen, C₁-C₁₂ alkyl, C₁-C₁₂ haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)₂, —NR¹⁰C(═O)NR¹⁰R¹¹, —C(═O)OR¹⁰, or —C(═O)NR¹⁰R¹¹;
each R⁴ and R⁵ are independently hydrogen or C₁-C₂ alkyl; and
each instance of R¹⁰ and R¹¹ independently is hydrogen or C₁-C₁₂ alkyl.

20. The method of claim 1, wherein the RICTOR inhibitor is of Formula II, and is bimiralisib.

21. The method of claim 1, wherein the RICTOR inhibitor is of Formula III and
X⁸is N and X⁵ and X⁶ are CH;
R¹² is NR¹⁸R¹⁹ wherein the R¹⁸ and R¹⁹ of NR¹⁸R¹⁹ together with the nitrogen to which they are attached form a C₃-C₂₀ heterocyclic ring containing one or more additional ring atoms N, O or S; and
R¹⁷ is aryl or heteroaryl, wherein the aryl and heteroaryl are optionally substituted with 1 or 2 substituents, each substituent independently is halogen, C₁-C₁₂ alkyl, C₁-C₁₂ haloalkyl, C₁-C₁₂ alkoxy, —NR¹⁰R¹¹, —OR¹⁰, —C(═O)R¹⁰, —NR¹⁰C(═O)R¹¹, —N(C(═O)R¹¹)₂, —NR¹¹C(═O)NR¹⁰R¹¹, —C(═O)OR¹⁰, or —C(═O)NR¹⁰R¹¹.

22. The method of claim 1, wherein the RICTOR inhibitor is of Formula III and is vistusertib.

23. The method of claim 1, wherein the RICTOR inhibitor is of Formula IV and is CID613034 (3-(3,4-Dichlorophenyl)-1-(5-methyl-2-thiazolin-2-yl)-1-(3,4-xylyl)urea).
